(19) United States
(12) Patent Application Publication
(10) Pub. No.: US 2011/0072537 A1
(43) Pub. Date: Mar. 24, 2011

(54) Genetically modified plants having desirable traits

(76) Inventor: Ousama M. Zaghmout, Lorton, VA (US)

(21) Appl. No.: 11/111,760

(22) Filed: Apr. 22, 2005

Related U.S. Application Data
(60) Provisional application No. 60/564,207, filed on Apr. 22, 2004.

(51) Int. Cl. C12N 15/11; A01H 5/00

(52) U.S. Cl. 800/279; 800/290; 800/298

(57) ABSTRACT

A method for genetically modifying a turfgrass plant cell such that a plant, produced from said cell, is characterized as having modulated brassinolide activity as compared with a wild-type plant, said method comprising: introducing at least one exogenous BAS1 polynucleotide, homologue or functional fragment thereof, into a plant cell to obtain a transformed plant cell; and growing the transformed plant cell under conditions which permit expression of BAS1 gene product, homologue or functional fragment thereof, thereby producing a plant having modulated brassinolide activity, wherein the plant cell is a turf grass cell wherein said genetically modified plant further comprising a drought resistance, salt resistance, insect resistance and the like.

### CROSS-REFERENCE TO RELATED APPLICATIONS

This application claims the benefit of U.S. Provisional Application No. 60/564,207, filed Apr. 22, 2004. The entire disclosure of this prior application is hereby incorporated by reference.

### STATEMENT REGARDING FEDERALLY SPONSORED RESEARCH OR DEVELOPMENT

Not applicable.

### FIELD OF THE INVENTION

The present invention relates to the transformation and regeneration of transformed plants. In particular, the invention relates to the to the transformation and regeneration of transformed turf grass plants which confer the modulation of dwarfism and dark-green leaves in adult plants, drought resistance, salt resistance, insect resistance and the like.

### BACKGROUND OF THE INVENTION

The objective in many turf management situations is to maintain a dense turf with active photosynthesis, leaf formation and tillering but with reduced leaf or stem elongation and prevention of inflorescence formation. For example, maintaining grass found on lawns, sport fields, playgrounds, parks, golf courses, roadsides and cemeteries is costly. Retarding the growth of the grass can reduce maintenance costs by, for example, allowing for a reduction in mowing. Moreover, retarding plant growth is desired for aesthetic reasons. For example, dwarf ornamental plants are aesthetically pleasing and represent a class of plants having significant commercial importance. Furthermore, it may be necessary to control the growth of plants for safety reasons. For example, it may be necessary to retard the growth of plants which are near power lines and railroad rights-of-way.

Reducing the shoot growth in a flowering plant is extremely useful in many circumstances. First it makes the plant more resistant to adverse weather conditions in the field, such as wind, rain, hail and snow. Secondly, it makes the plant more compact, more stocky, and more resistant to falling over (technically known as “lodging”) as a result of the aforementioned weather conditions and/or as a result of heavy fruit or seed or grain production. Thirdly, in orchard situations a more compact nature of the shrub or tree is extremely valuable for a variety of reasons, including ease of tending the tree, picking the fruit, applying other treatments and reducing the necessity to prune the tree or shrub. Also, shoot growth resulting either from the presence of high levels of endogenous gibberellins, or induced by gibberellins applied to the plant, can compete with growth and development of fruit, seed or grain, thereby reducing the final yield.

Several plant growth regulators, such as plant growth retardants, are commercially available. For example, N-phosphonocarbonyl carbamic acid derivatives, carboxyphosphonates, substituted 2,3-dihydro-1,4-oxathin-2,6-dimethylphenoxy, epoxycyclohexane, derivatives of acrylic acid and imidazoline compounds are known. However, use of many of these compounds is cost prohibitive. Moreover, many of these compounds are toxic. There is therefore a need for low-cost, non-toxic plant growth retardants and methods for retarding plants utilizing such retardants. The present invention addresses this need.

Suppression of turfgrass by various growth retarding chemicals has been widely reported both for warm- and cool-season grass mixtures. However, responses have not always been consistent, yellowing may result and the growth retardant used may not be environmentally acceptable. The type of growth suppression varies with the chemical used, maleic hydrazide and mefluidide causing shoot mortality with rapid proliferation of new shoots whilst while flurprimidol and paclobutrazol only suppress foliar growth. Seedhead suppression is also desirable in many circumstances. Similarly there are many and reported beneficial responses with imazethapyn, mefluidide and prinexapacethyl in centipede grass. In the case of CGA 163935, high doses were required and this led to foliar damage.

Another desirable plant trait in the turf management programs is, but is not limited to, insect resistance. For example, any chemical insecticides, particularly organophosphates and carbamates, are neurotoxic to a wide range of animals from honey bees to humans. The use of a number of them have been discontinued because of their toxic properties. Thus, scientists are currently seeking alternatives to the conventional chemical approach to crop pest management. One such approach is the investigation of plant-mediated methods and products, which are more environmentally friendly. Other desirable plant traits in the turf management programs are, but not limited to, disease resistance, viral resistance as well as nematode resistance

Another desirable plant trait in the turf management programs is, but is not limited to, salt tolerance. Salt accumulation causes the following damages: (1) accumulated salt lowers the water potential in soil to prevent plants from absorbing water; (2) the salt absorbed (penetrated) into plants disturbs their metabolism; (3) salt inhibits the absorption of other ions necessary for viability (Sato, F., Plant Cell Engineering, Supplement, “Environmental Problems and Phytobiotechnology”, pp. 33-39, 1994). Especially, the inhibition of water absorption causes plants to lose turgor pressure and close stoma. Thus, photosynthesis is deteriorated and growth is seriously inhibited. Plants have evolved various mechanisms to adapt themselves to such environments. In a simple adaptation model, plant cells keep an osmotic difference between the inside and outside of the cells in some way, and restore turgor by water absorption. A non-plant example of this action is seen in halobacteria, which keep an osmotic balance between the inside and outside by accumulating salt in the cells. In this case, however, it is difficult to adapt them to environmental (osmotic) changes, because intracellular metabolic enzymes per se need to be salt-tolerant. Therefore, a better adaptation mechanism is the synthesis of a specific compound called “compatible solute” for keeping an intracellular osmosis depending on extrinsic osmotic changes as many salt-tolerant plants do so (see for example: U.S. Pat. No. 6,281,412).

Another desirable plant trait in the turf management programs is, but is not limited to, drought tolerance. Drought is a significant problem in agriculture today. Over the last 40 years, for example, drought accounted for 74% of the total U.S. crop losses of corn (Agriculture, U.S. Department of, 1990. Agricultural Statistics. US Government Printing Office, Washington, D.C.). During these water deficit periods, the photosynthetic rate decreases with the ultimate result of loss in yield (Boyer, J. S., In: Water deficits and plant growth, T. T. Kozlowski (ed.)., Academic Press, New York., pp. 154-190 (1976)). If carried to an extreme, severe water deficits result in death of the plant. To sustain productivity under adverse environmental conditions, it is important to provide crops with a genetic basis for coping with water deficit, for example by breeding water retention and tolerance mechanisms into crops so that they can grow and yield under these adverse conditions.

Another desirable plant traits in the turf management programs, but not limited to, is freezing tolerance. Studies on the tolerance of plants to freezing have revealed the mechanisms that contribute to survival at very low temperatures. Both physiological and genetic analyses point to the membrane systems as important to this homeostasis. Extreme cold results in the disruption of membrane function and death (Steponkus, P. L. (1984) Annu. Rev. Plant Physiol. 35:543-84). Plants can increase their tolerance to extreme cold by prior exposure to low, but non-freezing temperatures, a phenomenon known as cold acclimation. A prominent feature of cold acclimation is an alteration of the membrane lipid composition and the accumulation of simple sugars and some hydrophilic peptides, both of which are thought to stabilize membranes to protect against freezing injury (Thomashow, M. F. (1999) Annu. Rev. Plant Physiol. 50:5,71-99). Genetic analysis has implicated the plasma membrane as important to thermotolerance. Mutants hypersensitive to freezing injury (e.g., sfr) have defects in the cryostability of the plasma membranes (Warren, G., R. McKown et al. (1996) Plant Physiol 111(4):1011-9).

Scientists have long used cross-breeding and hybridization techniques to provide plants having particular desired traits such as appearance and reduced stem elongation, increase resistance or tolerance to drought, salt, water stress, cold, freezing, thermal stress, insect, virus, nematode, fungi and the like in a plant cell or plant, but these techniques are at best lengthy, time-consuming processes which do not necessarily result in the achievement of a particular goal. The advent of genetic engineering, however, provided the opportunity to introduce genetic material directly into a plant, which, upon expression in the plant, would result in a desired effect.

### SUMMARY

The present invention provides a method to control dwarf adult stature, increase resistance or tolerance to drought, salt, water stress, cold, freezing, thermal stress, insect, virus, nematode, fungi and the like in a plant cell or plant, comprising introducing an expression cassette into the cells of a plant to yield transformed plant cells. The expression cassette used in creating these transgenic plants of the invention comprises a gene or functional fragment thereof which confers at least one desirable traits such as dwarf adult stature, increase resistance or tolerance to drought, salt, water stress, cold, freezing, thermal stress, insect, virus, nematode, fungi and the like, operably linked to a promoter functional in the plant cell. Upon the expression of at least one of the genes encoding at least of the desirable traits in the transformed plant cells to control dwarf adult stature, increase resistance or tolerance to drought, salt, water stress, cold, freezing, thermal stress, insect, virus, nematode, fungi and the like. Transgenic turf grass plant cell or plant containing one or more of these desirable genes are also part of the invention. Plants resulting from a cross with any of these transgenic plants that are encompassed by this invention are also part of this invention. Preferably, the invention provides a method to control dwarf adult stature in a plant or a plant cell wherein said plant or plant cell further comprising at least one gene or functional fragment thereof which confers upon expression an increase resistance or tolerance to drought, salt, water stress, cold, freezing, thermal stress, insect, virus, nematode, fungi and the like.

The present invention further provides a method to control dwarf adult stature, increase resistance or tolerance to drought, salt, water stress, cold, freezing, thermal stress, insect, virus, nematode, fungi and the like in a turf grass plant cell or turf grass plant, comprising introducing an expression cassette into the cells of a plant to yield transformed plant cells wherein the plant is from a family or subfamily selected from Bambosoideae, Arundinoideae, Pooideae, Phalaenopsis, Poaceae, Festucoideae, Chlorideae, Hordeae, Zoysieae, Agrostideae, Festuceae and Panicoideae.

The present invention further provides a method to control dwarf adult stature, increase resistance or tolerance to drought, salt, water stress, cold, freezing, thermal stress, insect, virus, nematode, fungi and the like in a turf grass plant cell or turf grass plant, comprising introducing an expression cassette into the cells of a turfgrass plant to yield transformed turfgrass plant cells wherein the plant is selected from the group consisting essentially of St, Augustinegrasses, bluegrasses, bentgrasses, bermudagrasses, buffalograsses, redtop, bahiagrasses, centipedegrass, zoysiagrasses, ryegrasses and fescues.

The present invention further provides an expression cassette comprising a gene or fragment thereof which confers at least one desirable traits such as dwarf adult stature, an increase resistance or tolerance to drought, salt, water stress, cold, freezing, thermal stress, insect, virus, nematode, fungi and the like, operably linked to a promoter functional in the turfgrass plant cell. Transgenic turf grass plant cell or turfgrass plant containing one or more of these desirable genes are also part of the invention.

The present invention also provides a recombinant expression vector comprising a brassinolide activity modulating gene or functional fragment thereof wherein said vector is used in a transformation method of a plant or a plant cell.

A preferred embodiment of this invention is a recombinant expression vector comprising a brassinolide activity modulating gene or functional fragment thereof wherein said vector is used in a transformation method of a turfgrass plant or a turfgrass plant cell.

In the more preferred embodiment of the invention, the recombinant expression vector which further comprises at least an additional gene or functional fragment thereof selected from the group consisting of a gene encoding glucose oxidase, a gene encoding citrate synthase, a gene encoding A-9 desaturase, a gene encoding A-11 desaturase, a gene encoding a plant homolog of the neutrophil NADPH oxidase, a gene encoding bacteriopsin, a gene encoding an antiviral protein, a gene encoding cytokinin oxidase, a cytokinin regulated gene, an auxin-regulated gene, a gene encoding oxalate oxidase, a gene encoding cholesterol oxidase, a gene encoding a dehydration protein, a gene encoding an antimicrobial CEMA and/or CEMA-related peptide, systemic acquired resistant genes, a gene encoding Bacillus thuringiensis δ-endotoxin, a gene encoding synthetically-modified B. thuringiensis a gene encoding δ-endotoxins, a gene encoding Bacillus thuringiensis Cry1C, a gene encoding an insecticidal protein toxins from Photorhabdus, a gene encoding glyphosate resistant EPSP synthase, a gene encoding protoporphyrinogen oxidase, a gene encoding thiroedoxin, anti-apoptosis gene, a BOS1 gene, a gene encoding a protein toxic to diabrotica insects, a gene encoding Bacillus thuringiensis delta-endotoxin, a gene encoding Bacillus thuringiensis CryET33 and CryET34 proteins, a gene encoding resistant 5-enolpyruvyl-3-phosphoshikimate synthetase, a glyphosate resistant gene, glufosinate resistant gene, a gene associated with nucleotide triphosphate transport, a gene encoding molybdenum cofactor sulfurase, a gene encoding an enzyme of the glycine betaine biosynthetic pathway, a gene encoding a dehydration regulation gene, a DIMBOA biosynthetic gene, 13-glucosidase gene, tryptophan gene, a gene encoding P450 reductase or cytochrome P450 enzyme, a bacterial mannitol-1-P dehydrogenase, a gene encoding an enzyme catalyzing the production of a polyol, a gene encoding an mt1D gene, a gene encoding anthranilate synthase, Bonsai gene, a gene encoding phospholipid binding protein, a gene encoding Bax inhibitor-1, a gene encoding metabolite transporter, a gene encoding molybdenum cofactor sulfurase, a gene encoding polyhydroxyalkanoate (e.g., 3-hydroxyacyl-acyl carrier protein thioesterase gene), an alcohol dehydrogenase gene, glutathione reductase gene, dehydroascorbate reductase gene, monodehydroascorbate reductase gene, a gene encoding mitochondrial alternative oxidase, a gene encoding NADH oxidase, a gene encoding NADPH oxidase, a gene encoding heat shock protein, a gene which reduces bioactive GA levels and the height of a plant, an acetohydroxyacid synthase gene, NIM1 genes, an amine oxidase gene, a gene which encodes phytochrome A, a gene which allows introducing proximity-conditional dwarfing to plants allowing them to be grown at high densities while maintaining good yields, gene which expresses a reactive oxygen species producing enzyme, peroxidase gene, polyphenol oxidase gene, germin-like oxalate oxidase, a gene encoding galactose oxidase, a gene encoding superoxide dismutatse, a gene encoding catalase, a gene encoding glutathion peroxidase, a gene encoding ascorbate peroxidase, a gene encoding oxalate decarboxylase, a gene encoding choline oxidase, tfdA gene, a gene encoding a cold tolerance polypeptide from the Wcor410 family, a gene encoding a polypeptide having S-adenosylmethionine:methionine S-methyltransferase, a gene encoding the enzyme isopentenyl transferase, choline monooxygenase, a gene encoding trehalose-6-phosphate, phytase (These additional genes and functional fragments are listed in Table 1).

This invention also provides a recombinant expression vector comprising a polynucleotide sequence encoding a dwarfism gene or functional fragment thereof wherein said vector is used in a transformation method of a plant. More preferably, in the transformation of a turfgrass plant. Most preferably where the vector further comprising at least a gene or fragment of those genes listed above wherein said vector is used in the transformation of a plant, most preferably in the transformation of a turfgrass plant.

This invention also provides a recombinant expression vector comprising a polynucleotide sequence encoding a DWARF AND LATE FLOWERING 2 (DLF2) or functional fragment thereof wherein said vector is used in a transformation method of a plant. More preferably, in the transformation of a turfgrass plant. Most preferably where the vector further comprising at least an additional gene or functional fragment of a gene of those genes listed in Table 1, wherein said vector is used in the transformation of a plant, most preferably in the transformation of a turfgrass plant.

This invention also provides a recombinant expression vector comprising a polynucleotide sequence encoding a cytochrome P450 hydroxylase modulating gene or functional fragment thereof wherein said vector is used in a transformation of a plant. More preferably, in the transformation of a turfgrass plant. Most preferably where the vector further comprising at least a gene or fragment of those genes listed above wherein said vector is used in the transformation of a plant, most preferably in the transformation of a turfgrass plant. All sequences that are listed in EP1209227A3 are also part of this invention to be used in accordance with this invention.

This invention also provides a recombinant expression vector comprising a polynucleotide sequence encoding a cytokinin oxidase or functional fragment thereof wherein said vector is used in a transformation of a plant. More preferably, in the transformation of a turfgrass plant. Most preferably wherein the vector further comprising at least an additional gene or functional fragment selected from the list in Table 1, wherein said vector is used in the transformation of a plant, most preferably in the transformation of a turfgrass plant.

More preferably, this invention provides the recombinant expression vector which further comprises an additional gene or a fragment thereof encoding for drought resistance, salt resistance, insect resistance and the like; wherein the additional gene or functional fragment of is selected from the list in Table 1.

More preferable, this invention provides a host cell containing the recombinant expression vector of any of the embodiments of the invention.

This invention further provides a genetically modified a turf grass plant comprising at least one of the recombinant expression vectors of any of the embodiments of the invention. More preferable, the plant contains multiple exogenous nucleic acid sequences encoding at least any of the polypeptides of the invention.

More preferably, this invention provides a genetically modified turf grass plant comprising at least one exogenous nucleic acid sequence encoding an BAS 1 polypeptide, homologue or functional fragment thereof, in its genome or at least one regulatory sequence that modifies expression of endogenous BAS1 gene, homologue or functional fragment thereof, and which is characterized as having modulated brassinolide activity or synthesis. More preferable, the plant contains multiple exogenous nucleic acid sequences encoding a BAS1 polypeptide. More preferable, the bas 1 nucleic acid sequence is operably associated with a regulatory nucleic acid sequence. More preferable, the regulatory nucleic acid sequence comprises a promoter. More preferable, the promoter is a constitutive promoter. More preferable, the promoter is an inducible promoter. More preferable, the promoter is induced by chemical means. More preferable, wherein the nucleic acid in the plant further comprises a selectable marker. More preferable, the turfgrass plant that are encompassed by the invention. Most preferable, the plant tissue is derived from these transgenic plants. More preferable, the seed that germinates into a plant comprising at least one exogenous BAS1 nucleic acid sequence, homologue or functional fragment thereof, in its genome and characterized as having modulated brassinolide activity, wherein the seed is a turf grass seed. More preferably, a seed that germinates into a plant that overexpresses and/or underexpress an endogenous bas 1 gene, homologue or functional fragment thereof, wherein the seed is a turf grass seed selected from the group that are encompassed by the invention. More preferable, a seed that results from the transformation with any of the expression vectors that is encompassed by this invention. Most preferable, a seed that results from a cross with a plant resulting from the transformation with any of the expression vectors that is encompassed by this invention. Most preferable, a seed that results from a sexually propagated plant wherein the plant is resulting from the transformation with any of the expression vectors that is encompassed by this invention. More preferably said plant or plant cell confers the modulation of dwarfism with dark-green leaves in adult plants and a gene which confers a drought resistance, salt resistance, insect resistance and the like.

This invention provides a method for genetically modifying a plant cell such that a plant, produced from said cell, is characterized as having modulated brassinolide activity as compared with a wild-type plant, said method comprising:

introducing at least one exogenous gene, homologue or functional fragment thereof, into a plant cell to obtain a transformed plant cell; and
growing the transformed plant cell under conditions which permit expression of said gene, homologue or functional fragment thereof,
thereby producing a plant having modulated brassinolide activity, wherein said genetically modified plant further comprises an additional gene or functional fragment thereof which confers drought resistance, salt resistance, insect resistance and the like. According to the invention, the additional gene or the additional functional fragments thereof which confers drought resistance, salt resistance, insect resistance and the like that is selected from those listed in the Table 1.

This invention provides a method for genetically modifying a turfgrass plant cell such that a plant, produced from said cell, is characterized as having modulated brassinolide activity as compared with a wild-type plant, said method comprising:

introducing at least one exogenous BAS 1 polynucleotide, homologue or functional fragment thereof, into a plant cell to obtain a transformed plant cell; and
growing the transformed plant cell under conditions which permit expression of BAS1 gene product, homologue or functional fragment thereof,
thereby producing a plant having modulated brassinolide activity, wherein the plant cell is a turf grass cell wherein said genetically modified plant further comprising an additional gene which confers to drought resistance, salt resistance, insect resistance and the like. According to the invention, the gene or the functional fragments thereof which confers drought resistance, salt resistance, insect resistance and the like is selected from the list of Table 1.

This invention provides a method for genetically modifying a turfgrass plant cell such that a plant, produced from said cell, is characterized as having modulated dwarfism gene activity as compared with a wild-type plant, said method comprising:

introducing at least one exogenous dwarfism gene, homologue or functional fragment thereof, into a plant cell to obtain a transformed plant cell; and
growing the transformed plant cell under conditions which permit expression of dwarfism gene product, homologue or functional fragment thereof,
thereby producing a plant having an alteration in stem and/or leaf length, wherein the plant cell is a turf grass cell wherein said genetically modified plant further comprises an additional gene which confers a drought resistance, salt resistance, insect resistance and the like. According to the invention, the additional gene or the additional functional fragments thereof which confers drought resistance, salt resistance, insect resistance and the like is selected from Table 1.

This invention provides a method for genetically modifying a turfgrass plant cell such that a plant, produced from said cell, is characterized as having modulated Late Flowering gene activity as compared with a wild-type plant, said method comprising:

introducing at least one exogenous Dwarf and Late Flowering gene, homologue or functional fragment thereof, into a plant cell to obtain a transformed plant cell; and
growing the transformed plant cell under conditions which permit expression of Dwarf and Late flowering gene product, homologue or functional fragment thereof,
thereby producing a plant having an alteration in time of flowering and/or stem and/or leaf length, wherein the plant cell is a turf grass cell wherein said genetically modified plant further comprising a drought resistance, salt resistance, insect resistance and the like. According to the invention, the additional gene or the additional functional fragment thereof which confers drought resistance, salt resistance, insect resistance and the like is selected from the list in Table 1.

This invention provides a method for genetically modifying a turfgrass plant cell such that a plant, produced from said cell, is characterized as having modulated DLF2 gene activity as compared with a wild-type plant, said method comprising:

introducing at least one exogenous DLF2 gene, homologue or functional fragment thereof, into a plant cell to obtain a transformed plant cell; and
growing the transformed plant cell under conditions which permit expression of DLF2 gene product, homologue or functional fragment thereof,
thereby producing a plant having an alteration in time of flowering and/or stem and/or leaf length, wherein the plant cell is a turf grass cell wherein said genetically modified plant further comprising an additional gene which confers drought resistance, salt resistance, insect resistance and the like. According to the invention, the additional gene or the additional functional fragments thereof which confers drought resistance, salt resistance, insect resistance and the like that is selected from the list of genes shown in Table 1.

This invention provides a method for genetically modifying a turfgrass plant cell such that a plant, produced from said cell, is characterized as having modulated cytochrome P450 hydroxylase gene activity as compared with a wild-type plant, said method comprising:

introducing at least one exogenous cytochrome P450 hydroxylase gene, homologue or functional fragment thereof, into a plant cell to obtain a transformed plant cell; and
growing the transformed plant cell under conditions which permit expression of cytochrome P450 hydroxylase gene product, homologue or functional fragment thereof,
thereby producing a plant having an alteration in stem and/or leaf length, wherein the plant cell is a turf grass cell wherein said genetically modified plant further comprising an additional gene which confers drought resistance, salt resistance, insect resistance and the like. According to the invention, the additional gene or the functional fragments thereof which confers drought resistance, salt resistance, insect resistance and the like is selected from list of genes shown in Table 1. Plants and plants produced from said method are also part of this invention.

This invention provides a method for genetically modifying a turfgrass plant cell such that a plant, produced from said cell, is characterized as having modulated cytokinin oxidase gene activity as compared with a wild-type plant, said method comprising:

introducing at least one exogenous dwarfism gene, homologue or functional fragment thereof, into a plant cell to obtain a transformed plant cell; and
growing the transformed plant cell under conditions which permit expression of cytokinin oxidase gene product, homologue or functional fragment thereof,
thereby producing a plant having an alteration in stem and/or leaf length, wherein the plant cell is a turf grass cell wherein said genetically modified plant further comprises an additional gene of a functional fragment thereof which confers upon expression drought resistance, salt resistance, insect resistance and the like. According to the invention, the additional gene or additional the functional fragments thereof which confers drought resistance, salt resistance, insect resistance and the like is selected from the list in Table 1.

According to the embodiments of the invention, the expression is overexpression and the modulation is dwarfism accompanied by dark-green turfgrass in adult plants.

According to the embodiments of the invention, the modulation is suppressed brassinolide activity.

According to the embodiments of the invention, suppression of brassinolide activity is achieved by overexpression or underexpression of BAS1 or a functional fragment thereof in the plant.

According to the embodiments of the invention, suppression of brassinolide activity is achieved by overexpression of dwarfism gene or a functional fragment thereof in the plant.

According to the embodiments of the invention, suppression of brassinolide activity is achieved by overexpression of Dwarf and Late flowering gene or a functional fragment thereof in the plant.

According to the embodiments of the invention, suppression of brassinolide activity is achieved by overexpression of DLF2 or a functional fragment thereof in the plant.

According to the embodiments of the invention, suppression of brassinolide activity is achieved by overexpression or underexpression of cytochrome P450 hydroxylase or a functional fragment thereof in the plant.

According to the embodiments of the invention, suppression of brassinolide activity is achieved by overexpression of cytokinin oxidase or a functional fragment thereof in the plant.

The method of this invention comprises introducing at least one exogenous polynucleotide comprising a chibi2 structural gene into the plant cell to obtain the transformed plant cell and growing the transformed plant cell under conditions which permit expression of the chibi2 gene product, wherein the plant cell is of a turfgrass plant origin wherein said transformed plant cell further comprises an additional gene or a functional fragment thereof which confers upon expression a desirable trait selected from the group consisting of a drought resistance, salt resistance, insect resistance and the like. It is understood that the invention is not limited to turfgrass plants, the invention does encompass also plant species other than turfgrass plants.

This invention provides a method of producing a genetically modified plant characterized as having dwarf adult stature with dark green foliage, said method comprising:

contacting a plant cell with a vector containing an exogenous nucleic acid sequence comprising at least one structural gene encoding a BAS1 polypeptide, homologue or functional fragment thereof, said gene being operably associated with a regulatory sequence that causes overexpression of the gene, to obtain a transformed plant cell;
producing a plant from said transformed plant cell; and
selecting a plant exhibiting said dwarf adult stature with dark green foliage; wherein said transformed plant cell further comprises a gene or a fragment thereof which confers upon expression a desirable trait selected from the group consisting of a drought resistance, salt resistance, insect resistance and the like.

This invention provides a method of producing a genetically modified turfgrass plant characterized as having dwarf adult stature with dark green foliage, said method comprising:

contacting a plant cell with a vector containing an exogenous nucleic acid sequence comprising at least one structural gene encoding a BAS1 polypeptide, homologue or functional fragment thereof, said gene being operably associated with a regulatory sequence that causes overexpression of the gene, to obtain a transformed plant cell;
producing a plant from said transformed plant cell; and
selecting a plant exhibiting said dwarf adult stature with dark green foliage, drought resistance, salt resistance, insect resistance and the like; wherein said transformed plant cell further comprises an additional gene or an additional fragment thereof that is selected from the list of genes in Table 1.

According to this invention, the contacting for performing transformation is by physical means.

According to this invention, the contacting for performing transformation is by chemical means.

According to the present invention, the plant cell is selected from the group consisting of protoplasts, gamete producing cells, and cells that regenerate into a whole plant.

According to the present invention, the regulatory sequence comprises a constitutive promoter.

According to the present invention, the regulatory sequence comprises an inducible promoter.

According to the present invention, plant, plant cell and plant tissue resulting from direct transformation with a gene of a fragment according to the embodiments of this invention are within the scope of the invention.

According to the present invention, plant, plant cell and plant tissue resulting from a cross with a transformed plant from a transformation according to the embodiments of this invention are within the scope of the invention.

The invention further provides a method for modulating brassinolide activity in a turfgrass plant comprising:

contacting a plant cell with the recombinant expression vector according to the embodiments of this invention to obtain a transformed plant cell;
growing the transformed plant cell under plant forming conditions to produce a plant from said transformed plant cell; and
selecting a plant exhibiting said modulated brassinolide activity.

This invention further provides a genetically modified turfgrass plant having a transgene increasing expression of BAS1 gene, homologue or functional fragment there of, chromosomally integrated into the genome of the plant.

wherein said transgenic plants further confer drought resistance, salt resistance, insect resistance and the like, wherein said genetically modified turfgrass plant further comprises a gene, homologue or functional fragment thereof selected from the list of genes in Table 1.

The invention further provides a method of producing a genetically modified turfgrass plant characterized as having increased resistance or tolerance to an insect as compared to the corresponding wild-type plant, said method comprising:

a) contacting plant cells with nucleic acid encoding a BAS 1 polypeptide, wherein said nucleic acid is operatively associated with an expression control sequence, to obtain transformed plant cells;
b) producing plants from said transformed plant cells under conditions which allow expression of BAS 1; and
c) selecting a plant exhibiting said resistance or tolerance wherein the genetically modified turfgrass plant further possesses drought resistance, salt resistance, fungus resistance, nematode resistance and the like wherein the genetically modified turfgrass plant further comprises an additional gene or an additional fragment thereof that is selected from the list in Table 1.

In one aspect of the invention, the modulation of dwarfism and dark-green leaves in adult plants, drought resistance, salt resistance, insect resistance and the like may be performed by contacting a cell with nucleic acid encoding a BSA 1 polypeptide and at least an additional gene or functional fragment thereof which confers the modulation of dwarfism and dark-green leaves in adult plants, drought resistance, salt resistance, insect resistance and the like wherein the additional gene or the additional fragment thereof is selected from list in Table 1.

According to the embodiments of this invention, the plant cell is selected form the group

consisting of protoplasts, gamete producing cells, and cells which regenerate into whole plants.

In one aspect of the invention, the resistance to pathogen is increasing resistance to a bacterial pathogen.

This invention further provides a method of producing a turfgrass plant characterized as having increased disease or insect resistance or tolerance as compared to a wild-type plant, said method comprising contacting a susceptible plant with a BAS 1 promoter-inducing amount of an agent necessary to elevate BAS 1 gene expression above BAS 1 expression in a plant not contacted with the agent, wherein said transgenic plant further comprising at least an additional gene, homologue or functional fragment thereof which confers upon expression drought resistance, salt resistance, insect resistance and the like wherein the additional gene, homologue or functional fragment thereof is selected from the list of genes in Table 1. In one aspect of this invention, the agent is a transcription factor, a chemical agent, chemical enhancer or chemical inducer. Examples of these of these chemicals are listed in U.S. Pat. No. 5,942,662, EP0540561, EP0392225, U.S. Pat. No. 5,877,400, WO02071834, WO03030821, US20020129399, US20020188965, WO02085104 where the full content of these issued patents and published patent applications are incorporated in full in this invention.

The invention further provides a method of producing genetically transformed turfgrass plant, which is a disease-resistant plants, comprising introducing into the genome of a plant cell to obtain a transformed plant cell, a nucleic acid sequence comprising an expression control sequence operably linked to a polynucleotide encoding BAS 1 polypeptide, wherein said transgenic plants further comprises at least an additional gene, homologue or functional fragment thereof which confers upon expression drought resistance, salt resistance, insect resistance and the like wherein the additional gene, homologue or functional fragment thereof is selected from the list in Table 1.

In one aspect of this invention, the expression control sequence targets expression to a plant tissue selected from the group consisting of leaves, roots, shoots, and stems. In one aspect of this invention, the agent is a transcription factor, a chemical agent, chemical inducer, or chemical enhancer.

The invention further provides a plant produced by the methods of this invention, plant tissue derived from a plant produced by the methods of this invention, and a seed as well as harvestable parts or propagation material derived from a plant produced by the methods of this invention.

In yet other embodiments of this invention, Bas 1 gene, homologue, functional fragment thereof is replaced in certain aspect of this invention with other genes such as DWARIFISM gene, Dwarf Late Flowering gene, DFL2, cytochrome P450 hydroxylase, or cytokinin oxidase, their homologues, or their functional fragments thereof.

In yet other embodiments of this invention, transgenic plants including, but not limited to, monocot and dicot plant species that have been transformed with the recombinant expression vectors of this invention are also part of this invention. More preferably, transgenic turf grass plants that are transformed with recombinant expression vector of this invention are also part of this invention, whether the recombinant expression vector contain one or more of the genes, homologues or fragments thereof which confer dwarfism and dark-green leaves in adult plants, a drought resistance, salt resistance, insect resistance and the like.

In yet another embodiment of the invention is a method of regenerating, in vitro or in vivo, a mature fertile transgenic or not transgenic plant through direct organogenesis, said method comprising: cytokinin or compounds with cytokinin activities.

In yet another embodiment of the invention is a method of regenerating, in vitro or in vivo, a mature fertile transgenic or not transgenic plant through direct organogenesis, said method comprising:

cytokinin or compounds with cytokinin activities such as, but not limited to, kinetin, zeatin, aminopurine, thidiazuron and a like wherein the plant can be any of the plants species mentioned in this application and other plants species that are known to those of ordinary skills in the art. Part of this invention is a method of transforming a plant or a plant cell using a plant or a plant without a root, leaf, or shoot; or a plant without substantial amount of roots, leaves, or shoots.
What is claimed is:
1. A recombinant expression vector comprising a polynucleotide sequence encoding a BAS 1 polypeptide or functional fragment thereof.
2. A host cell containing the vector of claim 1.
3. A recombinant expression vector according to claim 1 further comprising at least an additional gene, a homologue, or functional fragment thereof that is selected from the group consisting of a gene encoding glucose oxidase, a gene encoding citrate synthase, a gene encoding A-9 desaturase, a gene encoding A-11 desaturase, a gene encoding a plant homolog of the neutrophil NADPH oxidase, a gene encoding bacteriopsin, a gene encoding an antiviral protein, a gene encoding cytokinin oxidase, a cytokinin regulated gene, an auxin-regulated gene, a gene encoding oxalate oxidase, a gene encoding cholesterol oxidase, a gene encoding for dehydration protein, a gene encoding an antimicrobial CEMA and/or CEMA-related peptides, acquired resistant genes, Bacillus thuringiensis δ-endotoxins, synthetically-modified B. thuringiensis a gene segments encoding δ-endotoxins, a gene encoding Bacillus thuringiensis Cry1C, a gene encoding an insecticidal protein toxins from Photorhabdus, a gene encoding glyphosate resistant EPSP synthase, a gene encoding protoporphyrinogen oxidase, a gene encoding THIOREDOXIN, a ANTI-APOPTOSIS GENE, a BOS1 gene, a gene encoding a protein toxic to diabrotica insects, a gene encoding Bacillus thuringiensis delta-endotoxin, a gene encoding Bacillus thuringiensis CryET33 and CryET34 proteins, a gene encoding resistant 5-enolpyruvyl-3-phosphoshikimate synthetase, a glyphosate resistant gene, glufosinate resistant gene, a gene associated with nucleotide triphosphate transport, a gene encoding molybdenum cofactor sulfurase, a gene encoding an enzyme of the glycine betaine biosynthetic pathway, DNA and encoded protein which regulates dehydration regulated genes, a DIMBOA biosynthesis genes, 13-glucosidase gene, a tryptophan gene, a gene encoding P450 reductases or cytochrome P450 enzymes, a bacterial mannitol-1-P dehydrogenase, a gene encoding an enzyme catalyzing the production of a polyol, a gene encoding an mt1D gene, a gene encoding maize anthranilate synthase, Bonsai, a gene encoding phospholipid binding protein, a gene encoding Bax inhibitor-1, a gene encoding metabolite transporters, a gene encoding molybdenum cofactor sulfurase, a gene encoding polyhydroxyalkanoate (e.g., 3-hydroxyacyl-acyl carrier protein thioesterase gene), an alcohol dehydrogenase gene, glutathione reductase gene, dehydroascorbate reductase gene, monodehydroascorbate reductase gene, a gene encoding mitochondrial alternative oxidase, a gene encoding NADH oxidase, a gene encoding NADPH oxidase, a gene encoding heat shock protein, a gene which reduces bioactive GA levels and the height of a plant, dwarfism genes, an acetohydroxyacid synthase gene, NIM1 genes (NIM1 gene which allows activation in plant of systemic acquired resistance—useful to confer broad spectrum disease resistance in plants, specifically crop plants, e.g. rice, wheat, barley, rye and corn), an amine oxidase gene, coding sequence which encodes phytochrome A, a gene which allows introducing proximity-conditional dwarfing to plants allowing them to be grown at high densities while maintaining good yields, gene which expresses a reactive oxygen species producing enzyme), peroxidase gene, polyphenol oxidase gene, germin-like oxalate oxidase gene, oxalate (oxalic) oxidase gene, a gene encoding galactose oxidase, a gene encoding superoxide dismutatse, a gene encoding catalase, a gene encoding glutathion peroxidase, a gene encoding ascorbate peroxidase, a gene encoding oxalate decarboxylase, a gene encoding choline oxidase, tfdA gene, a gene encoding a cold tolerance polypeptide from the Wcor410 family, a gene encoding a polypeptide having S-adenosylmethionine:methionine S-methyltransferase, gene encoding the enzyme isopentenyl transferase, choline monooxygenase, and phytase.
4. A genetically modified plant comprising at least one exogenous nucleic acid sequence encoding an BAS 1 polypeptide, homologue or functional fragment thereof, in its genome or at least one regulatory sequence that modifies expression of endogenous bas1 gene, homologue or functional fragment thereof, and which is characterized as having modulated brassinolide activity or synthesis.
5. The plant of claim 4, wherein the plant contains multiple exogenous nucleic acid sequences encoding a BAS 1 polypeptide.
6. A recombinant expression vector comprising a polynucleotide sequence encoding a BAS 1 polypeptide or functional fragment thereof wherein said vector is used in a transforming a turfgrass plant.
7. The plant of claim 4, wherein the modulation is dwarfism with dark-green leaves in adult plants.
8. A recombinant expression vector comprising a polynucleotide sequence encoding a DWARFISM GENE or functional fragment thereof wherein said vector is used in a transformation method of a turfgrass plant.
9. The plant of claim 4, wherein the bas 1 nucleic acid sequence is operably associated with a regulatory nucleic acid sequence.
10. The plant of claim 9, wherein the regulatory nucleic acid sequence comprises a promoter.
11. The plant of claim 10, wherein the promoter is a constitutive promoter.
12. The plant of claim 10, wherein the promoter is an inducible promoter. The plant of claim 10, wherein the promoter is induced by chemical means.
13. The plant of claim 4, wherein the nucleic acid further comprises a selectable marker.
14. The plant of claim 4 wherein the regulatory sequence that modifies expression of endogenous bas 1 gene, homologue or functional fragment thereof, comprises a transfer DNA containing four or more copies of enhancer regions from the CaMV 35S promoter.
15. The plant of claim 4, wherein the plant is a monocotyledonous plant.
16. The plant of claim 4, wherein the monocotyledonous plant is a turfgrass or a forage grass plant.
17. Plant tissue derived from the plant of claim 4.
18. A seed that germinates into a plant comprising at least one exogenous BAS1 nucleic acid sequence, homologue or functional fragment thereof, in its genome and characterized as having modulated brassinolide activity, wherein the seed is a turf grass seed.
19. A seed that germinates into a plant that overexpresses an endogenous bas 1 gene, homologue or functional fragment thereof, wherein the seed is a turf grass seed.
20. A vector containing at least one exogenous nucleic acid sequence encoding a BAS1 polypeptide, homologue or functional fragment thereof, operably associated with a promoter wherein said vector further comprises a gene which confers, wherein the gene is selected from the group consisting of:
21. A recombinant expression vector comprising a polynucleotide sequence encoding a DWARF AND LATE FLOWERING 2 (DLF2) or functional fragment thereof wherein said vector is used in a transformation method of a turfgrass plant.
22. A recombinant expression vector comprising a polynucleotide sequence encoding a Cytochrome P450 hydroxylase modulating gene or functional fragment thereof wherein said vector is used in a transformation method of a turfgrass plant.
23. A recombinant expression vector comprising a polynucleotide sequence encoding a brassinolide activity modulating gene or functional fragment thereof wherein said vector is used in a transformation method of a turfgrass where is said gene or said functional fragment thereof is selected from a group consisting of BAS 1 and GA3 oxidase.
24. The recombinant expression vector according to claim 1 further comprising a gene encoding for drought resistance, salt resistance, insect resistance and the like.
25. The recombinant expression vector according to claim 1 further comprising a gene encoding for drought resistance, salt resistance, insect resistance and the like wherein the gene is selected from the genes or fragments thereof listed in Table 1.
26. A method for genetically modifying a turfgrass plant cell such that a plant, produced from said cell, is characterized as having modulated brassinolide activity as compared with a wild-type plant, said method comprising:
introducing at least one exogenous BAS1 polynucleotide, homologue or functional fragment thereof, into a plant cell to obtain a transformed plant cell; and
growing the transformed plant cell under conditions which permit expression of BAS1 gene product, homologue or functional fragment thereof,
thereby producing a plant having modulated brassinolide activity, wherein the plant cell is a turf grass cell wherein said genetically modified plant further comprising a drought resistance, salt resistance, insect resistance and the like.
28. The method of claim 26, wherein the expression is overexpression and the modulation is dwarfism accompanied by dark-green turfgrass in adult plants.
29. The method of claim 26, wherein the expression is overexpression and the modulation is dwarfism accompanied by dark-green turfgrass in adult plants wherein the turfgrass plant is a bentgrass, kentucky bluegrass, bermudagrass, zosiagrass, fescues, ryegrass.
30. The method of claim 26, wherein said modulation is suppressed brassinolide activity.
31. The method of claim 24, wherein said suppression of brassinolide activity is achieved by overexpression of BAS1 in the plant.
32. The method of claim 24, further comprising introducing at least one exogenous polynucleotide comprising a chibi2 structural gene into the plant cell to obtain the transformed plant cell and growing the transformed plant cell under conditions which permit expression of the chibi2 gene product, wherein the plant cell is a turfgrass plant origin.
33. A method of producing a genetically modified plant characterized as having dwarf adult stature with dark green turfgrass plant, said method comprising:
contacting a plant cell with a vector containing an exogenous nucleic acid sequence comprising at least one structural gene encoding a BAS1 polypeptide, homologue or functional fragment thereof, said gene being operably associated with a regulatory sequence that causes overexpression of the gene, to obtain a transformed plant cell;
producing a plant from said transformed plant cell; and
selecting a plant exhibiting said dwarf adult stature with dark green foliage.
34. The method of claim 33, wherein the contacting is by physical means.
35. The method of claim 33, wherein the contacting is by chemical means.
36. The method of claim 33, wherein the plant cell is selected from the group consisting of protoplasts, gamete producing cells, and cells that regenerate into a whole plant.
37. The method of claim 33, wherein the regulatory sequence comprises a constitutive promoter.
38. The method of claim 33, wherein the regulatory sequence comprises an inducible promoter.
39. A plant produced by the method of claim 33.
40. Plant tissue derived from a plant produced by the method of claim 33.
41. A method for modulating brassinolide activity in a turfgras plant comprising: contacting a plant cell with the vector of claim 20 to obtain a transformed plant cell; growing the transformed plant cell under plant forming conditions to produce a plant from said transformed plant cell; and selecting a plant exhibiting said modulated brassinolide activity.
42. A genetically modified turfgrass plant having a transgene increasing expression of BAS1 gene, homologue or functional fragment thereof, chromosomally integrated into the genome of the plant wherein said transgenic plants further comprising a drought resistance, salt resistance, insect resistance and the like.
43. A host cell containing the vector of claims 1-8.
44. A genetically modified a turf grass plant comprising at least one exogenous nucleic acid sequence encoding an BAS 1 polypeptide, homologue or functional fragment thereof, in its genome or at least one regulatory sequence that modifies expression of endogenous BAS1 gene, homologue or functional fragment thereof, and which is characterized as having modulated brassinolide activity or synthesis.
45. A modified plant comprising at least one exogenous nucleic acid sequence encoding an BAS 1 polypeptide, homologue or functional fragment thereof, in its genome or at least one regulatory sequence that modifies expression of endogenous BAS1 gene, homologue or functional fragment thereof, and which is characterized as having modulated brassinolide activity or synthesis wherein said transgenic plants further comprising a drought resistance, salt resistance, insect resistance and the like.
46. A method of producing a genetically modified turfgrass plant characterized as having dwarf adult stature with dark green foliage, said method comprising:
contacting a plant cell with a vector containing an exogenous nucleic acid sequence comprising at least one structural gene encoding a BAS 1 polypeptide, homologue or functional fragment thereof, said gene being operably associated with a regulatory sequence that causes overexpression of the gene, to obtain a transformed plant cell;
producing a plant from said transformed plant cell; and
selecting a plant exhibiting said dwarf adult stature with dark green foliage wherein said transgenic plants further comprising a drought resistance, salt resistance, insect resistance and the like.
47. A method of producing a genetically modified turfgrass plant characterized as having dwarf adult stature with dark green foliage, said method comprising:
contacting a plant cell with a vector containing an exogenous nucleic acid sequence comprising at least one structural gene encoding a BAS 1 polypeptide, homologue or functional fragment thereof, said gene being operably associated with a regulatory sequence that causes overexpression of the gene, to obtain a transformed plant cell;
producing a plant from said transformed plant cell; and
selecting a plant exhibiting said dwarf adult stature with dark green foliage wherein said transgenic plants further comprising a drought resistance and the like.
48. A method of producing a genetically modified turfgrass plant characterized as having dwarf adult stature with dark green foliage, said method comprising:
contacting a plant cell with a vector containing an exogenous nucleic acid sequence comprising at least one structural gene encoding a BAS 1 polypeptide, homologue or functional fragment thereof, said gene being operably associated with a regulatory sequence that causes overexpression of the gene, to obtain a transformed plant cell;
producing a plant from said transformed plant cell; and
selecting a plant exhibiting said dwarf adult stature with dark green foliage wherein said transgenic plants further comprising a salt resistance and the like.
49. A method of producing a genetically modified turfgrass plant characterized as having dwarf adult stature with dark green foliage, said method comprising:
contacting a plant cell with a vector containing an exogenous nucleic acid sequence comprising at least one structural gene encoding a BAS 1 polypeptide, homologue or functional fragment thereof, said gene being operably associated with a regulatory sequence that causes overexpression of the gene, to obtain a transformed plant cell;
producing a plant from said transformed plant cell; and
selecting a plant exhibiting said dwarf adult stature with dark green foliage wherein said transgenic plants further comprising an insect resistance and the like.
50. A method of producing a genetically modified turfgrass plant characterized as having dwarf adult stature with dark green foliage, said method comprising:
contacting a plant cell with a vector containing an exogenous nucleic acid sequence comprising at least one structural gene encoding a BAS 1 polypeptide, homologue or functional fragment thereof, said gene being operably associated with a regulatory sequence that causes overexpression of the gene, to obtain a transformed plant cell;
producing a plant from said transformed plant cell; and
selecting a plant exhibiting said dwarf adult stature with dark green foliage wherein said transgenic plants further comprising a fungi resistance and the like.
51. A method of producing a genetically modified turfgrass plant characterized as having increased insect disease or insect as compared to the corresponding wild-type plant, said method comprising:
a) contacting plant cells with nucleic acid encoding a BAS 1 polypeptide, wherein said nucleic acid is operatively associated with an expression control sequence, to obtain transformed plant cells;
b) producing plants from said transformed plant cells under conditions which allow expression of BAS 1; and
c) selecting a plant exhibiting said disease or insect.
wherein said transgenic plants further comprising a drought resistance, salt resistance, insect resistance and the like.
52. The method of claim 51, wherein said increased disease or insect is increased resistance to a bacterial pathogen.
53. The method of claim 52, wherein said bacterial pathogen is selected from the group listed in the specification.
54. The method of claim 51, wherein the expression control sequence is a promoter.
55. The method of claim 51, wherein the contacting is by physical means.
56. The method of claim 51 wherein the contacting is by chemical means.
57. The method of claim 51, wherein the plant cell is selected form the group consisting of protoplasts, gamete producing cells, and cells which regenerate into whole plants.
58. The method of claim 51, wherein said nucleic acid is contained in a T-DNA derived vector.
59. A plant produced by the method of claim 51.
60. Plant tissue derived from a plant of claim 59.
61. A seed derived from a plant of claim 59.
62. A method for genetically modifying a transgenic turfgrass plant cell such that a plant, produced from said cell, is characterized as having increased disease or insect as compared with a wild-type plant, said method comprising:
a) introducing a BAS 1 polynucleotide into a plant cell to obtain a transformed plant cell; and
b) growing said transformed plant cell under conditions which permit expression of BAS 1 polypeptide thereby producing a plant having increased disease or insect.
wherein said transgenic plants further comprising a drought resistance, salt resistance, insect resistance and the like.
63. The method of claim 62, wherein said increased disease or insect is increased resistance to a bacterial pathogen.
64. The method of claim 63, wherein said bacterial pathogen is selected from the group listed in the specification.
65. A method of producing a turfgrass plant characterized as having increased disease or insect as compared to a wild-type plant, said method comprising contacting a susceptible plant with a BAS 1 promoter-inducing amount of an agent necessary to elevate BAS 1 gene expression above BAS 1 expression in a plant not contacted with the agent.
wherein said transgenic plants further comprising a drought resistance, salt resistance, insect resistance and the like.
66. The method of claim 65, wherein the agent is a transcription factor.
67. The method of claim 65, wherein the agent is a chemical agent.
68. The method of claim 65, wherein said increased disease or insect is increased resistance to a bacterial pathogen.
69. The method of claim 68, wherein said bacterial pathogen is selected from the group listed in the specification.
70. A method of producing genetically transformed turfgrass plant, disease-resistant plants,
comprising introducing into the genome of a plant cell to obtain a transformed plant cell, a nucleic acid sequence comprising an expression control sequence operably linked to a polynucleotide encoding BAS 1 polypeptide.
wherein said transgenic plants further comprising a drought resistance, salt resistance, insect resistance and the like.
71. The method of claim 70, wherein said expression control sequence targets expression to a plant tissue selected from the group consisting of leaves, roots, shoots, and stems.
72. The method of claim 70, wherein said disease or insect is resistance to a bacterial pathogen.
73. The method of claim 73, wherein said bacterial pathogen is selected from the group listed in the specification.
74. A plant produced by the method of claim 70.
75. Plant tissue derived from a plant produced by the method of claim 70.
76. A seed derived from a plant produced by the method of claim 70.
79. An embryo cell from the transgenic plant produced according any of the claims of this invention.
80. A callus cell from the transgenic plant produced according any of the claims of this invention.
81. A protoplast from the transgenic plant produced according any of the claims of this invention.
82. A fruit from the transgenic plant produced according any of the claims of this invention.
83. A vegetable from the transgenic plant produced according any of the claims of this invention.
84. A seed from the transgenic plant produced according any of the claims of this invention.
85. A leaf from the transgenic plant produced according any of the claims of this invention.
86 A meristem from the transgenic plant produced according any of the claims of this invention.

### DESCRIPTION OF THE PREFERRED EMBODIMENTS

The present invention will now be described more fully hereinafter with reference to the accompanying specification, in which preferred embodiments of the invention are described. This invention may, however, be embodied in different forms and should not be construed as limited to the specific embodiments set forth herein. Rather, these embodiments are provided so that this disclosure will be thorough and complete, and will fully convey the scope of the invention to those skilled in the art. The terminology used in the description of the invention herein is for the purpose of describing particular embodiments only and is not intended to be limiting of the invention. As used in the description of the invention and the appended claims, the singular forms “a”, “an” and “the” are intended to include the plural forms as well, unless the context clearly indicates otherwise.

The present invention provides methods for producing a plant with one or more nucleic acids for the acquisition of, or an improvement in, a property or characteristic of the plant and which would be useful in conferring upon plants dwarf adult stature with dark green foliage, a drought resistance, salt resistance, insect resistance and the like.

The methods involve using more than one gene that, when present in a plant, confers upon plants dwarf adult stature with dark green foliage, a drought resistance, salt resistance, insect resistance and the like. The invention provides significant advantages over previously used methods for optimization in a plant the dwarf adult stature with dark green foliage, a drought resistance, salt resistance, insect resistance and the like.

Unless otherwise defined, all technical and scientific terms used herein have the same meaning as commonly understood by one of ordinary skill in the art to which this invention belongs. All publications, patent applications, patents, and other references mentioned herein are incorporated by reference in their entirety. Except as otherwise indicated, standard methods may be used for the production of cloned genes, expression cassettes, vectors (e.g., plasmids), proteins and protein fragments according to the present invention. Such techniques are known to those skilled in the art. See e.g., J. Sambrook et al., Molecular Cloning: A Laboratory Manual Second Edition (Cold Spring Harbor Laboratory, Cold Spring Harbor, N.Y., 1989), and F. M. Ausubel et al., Current Protocols In Molecular Biology (Green Publishing Associates, Inc. and Wiley-Interscience, New York, 1991); J. Draper et al., eds., Plant Genetic Transformation And Gene Expression: A Laboratory Manual, (Blackwell Scientific Publications, 1988); and S. B. Gelvin & R. A. Schilperoort, eds., Introduction, Expression, And Analysis Of Gene Production In Plants.

In the description that follows, a number of terms are used extensively. The following definitions are provided to facilitate understanding of the invention.

A structural gene is a region of DNA having a sequence that is transcribed into messenger RNA (mRNA) that is then translated into a sequence of amino acids characteristic of a specific polypeptide. Structural genes also include gene encoding RNA products directly such as genes encoding transfer RNA (tRNA).

Promoters useful in the invention include both constitutive and inducible natural promoters as well as engineered promoters. The CaMV promoters are examples of constitutive promoters. To be most useful, an inducible promoter should 1) provide low expression in the absence of the inducer; 2) provide high expression in the presence of the inducer; 3) use an induction scheme that does not interfere with the normal physiology of the plant; and 4) have no effect on the expression of other genes. Examples of inducible promoters useful in plants include those induced by chemical means, such as the yeast metallothionein promoter, which is activated by copper ions (Mett, et al., Proc. Natl. Acad. Sci., U.S.A., 90: 4567, 1993); In2-1 and In2-2 regulator sequences, which are activated by substituted benzenesulfonamides, e.g., herbicide safeners (Hershey, et al., Plant Mol. Biol., 17: the GRE regulatory sequences, which are induced by glucocorticoids (Schena, et al., Proc. Natl. Acad. Sci., U.S.A., 88: 10421, 1991); and ethanol-inducible promoters (Caddick et al., supra). Other promoters (both constitutive and inducible) and enhancers will be known to those of skill in the art.

The particular promoter selected should be capable of causing sufficient expression to result in the overexpression of the structural gene product, e.g., BAS1, to decrease brassinolide synthesis or signaling. Decreased brassinolide synthesis or signaling is characterized by hyperresponsiveness to brassinolide in a light-dependent manner, the presence of hypocotyls that are longer than the wild type, and reduced sensitivity to a variety of light conditions compared to wild type plants. The promoters used in the vector constructs of the present invention may be modified, if desired, to affect their control characteristics. In a preferred approach, multimerized copies of enhancer elements from the cauliflower mosaic virus (CaMV) 35S promoter are incorporated near (e.g., within 381 nucleotides) 5′ to the start of the BAS1 gene.

When these enhancers are inserted near a gene, its transcription can be enhanced.

Tissue specific promoters may also be utilized in the present invention. As used herein, the term “tissue-specific promoter” means a DNA sequence that serves as a promoter, i.e., regulates expression of a selected DNA sequence operably linked to the promoter. A tissue-specific promoter effects expression of the selected DNA sequence in specific cells, e.g., in the root or in the shoot of a plant. The term also covers so-called “leaky” promoters, which regulate expression of a selected DNA primarily in one tissue, but cause expression in other tissues as well. Such promoters also may include additional DNA sequences that are necessary for expression, such as introns and enhancer sequences. An example of a tissue specific promoter is the HHA promoter expressed in shoot meristems (Atanassova, et al., Plant J., ˜: 291, 1992).

Other tissue specific promoters useful in transgenic plants, including the cdc2a promoter and cyc07 promoter, will be known to those of skill in the art. (See for example, Ito, et al., Plant Mol. Biol., 24: 863, 1994; Martinez, et al., Proc. Natl. Acad. Sci. USA, 89: 7360, 1992; Medford, et al., Plant Cell, 3: 359, 1991; Terada, et al., Plant Journal, 3: 241, 1993; Wissenbach, et al., Plant Journal, 4: 411, 1993). Examples of tissue specific promoters active in floral meristems are the promoters of the apetala 3 and apetala 1 genes which are described in Jack et al., Cell, 76: 703, 1994 and Hempel et al., Development, 124: 3845, 1997. In addition, a meristem-specific promoter from the UFO gene (U.S. Pat. No. 5,880,330) may be useful in the practice of the inventors.

Complementary DNA (cDNA) is a single-stranded DNA molecule that is formed from an mRNA template by the enzyme reverse transcriptase. Typically, a primer complementary to portions of mRNA is employed for the initiation of reverse transcription. Those skilled in the art also use the term “cDNA” to refer to a double-stranded DNA molecule consisting of such a single-stranded DNA molecule and its complementary DNA strand.

The term expression refers to the biosynthesis of a gene product. For example, in the case of a structural gene, expression involves transcription of the structural gene into mRNA and the translation of mRNA into protein.

A vector is a DNA molecule, such as a plasmid, cosmid, or bacteriophage, that has the capability of replicating autonomously in a host cell. Vectors typically contain one or a small number of restriction endonuclease recognition sites at which exogenous DNA sequences can be inserted in a determinable fashion without loss of an essential biological function of the vector, as well as a marker gene that is suitable for use in the identification and selection of cells transformed with the cloning vector. Many examples of suitable marker genes are known to the art and can be employed in the practice of the invention. The selectable marker gene may be the only heterologous gene expressed by a transformed cell, or may be expressed in addition to another heterologous gene transformed into and expressed in the transformed cell. Selectable marker genes are utilized for the identification and selection of transformed cells or tissues. Selectable marker genes include genes encoding antibiotic resistance, such as those encoding neomycin phosphotransferase II (NEO) and hygromycin phosphotransferase (HPT), as well as genes conferring resistance to herbicidal compounds. Herbicide resistance genes generally code for a modified target protein insensitive to the herbicide or for an enzyme that degrades or detoxifies the herbicide in the plant before it can act. See, DeBlock et al., EMBO J. 6, 2513 (1987); DeBlock et al., Plant Physiol. 91, 691 (1989); Fromm et al., Bio Technology 8, 833 (1990); Gordon-Kamm et al., Plant Cell 2, 603 (1990). For example, resistance to glyphosphate or sulfonylurea herbicides has been obtained using genes coding for the mutant target enzymes, 5-enolpyruvylshikimate-3-phosphate synthase (EPSPS) and acetolactate synthase (ALS). Resistance to glufosinate ammonium, boromoxynil, and 2,4-dichlorophenoxyacetate (2,4-D) have been obtained by using bacterial genes encoding phosphinothricin acetyltransferase, a nitrilase, or a 2,4-dichlorophenoxyacetate monooxygenase, which detoxify the respective herbicides. Selectable marker genes include, but are not limited to, genes encoding: neomycin phosphotransferase II (Fraley et al., CRC Critical Reviews in Plant Science 4, 1 (1986)); cyanamide hydratase (Maier-Greiner et al., Proc. Natl. Acad. Sci. USA 88, 4250 (1991)); aspartate kinase; dihydrodipicolinate synthase (Perl et al., BioTechnology 11, 715 (1993)); bar gene (Toki et al., Plant Physiol. 100,1503 (1992); Meagher et al., Crop Sci. 36,1367 (1996)); tryptophane decarboxylase (Goddijn et al., Plant Mol. Biol. 22, 907 (1993)); neomycin phosphotransferase (NEO; Southern et al., J. Mol. Appl. Gen. 1, 327 (1982)); hygromycin phosphotransferase (HPT or HYG; Shimizu et al., Mol. Cell. Biol. 6, 1074 (1986)); dihydrofolate reductase (DHFR); phosphinothricin acetyltransferase (DeBlock et al., EMBO J. 6, 2513 (1987)); 2,2-dichloropropionic acid dehalogenase (Buchanan-Wollatron et al., J. Cell. Biochem. 13D, 330 (1989)); acetohydroxyacid synthase (U.S. Pat. No. 4,761,373 to Anderson et al.; Haughn et al., Mol. Gen. Genet. 221, 266 (1988)); 5-enolpyruvyl-shikimate-phosphate synthase (aroA; Comai et al., Nature 317, 741 (1985)); haloaryinitrilase (WO 87/04181 to Stalker et al.); acetyl-coenzyme A carboxylase (Parker et al., Plant Physiol. 92,1220 (1990)); dihydropteroate synthase (sull; Guerineau et al., Plant Mol. Biol. 15, 127 (1990)); and 32 kDa photosystem II polypeptide (psbA; Hirschberg et al., Science 222,1346 (1983)). Also included are genes encoding resistance to chloramphenicol (Herrera-Estrella et al., EMBO J. 2, 987 (1983)); methotrexate (Herrera-Estrella et al., Nature 303, 209 (1983); Meijer et al., Plant Mol. Biol. 16, 807 (1991)); hygromycin (Waldron et al., Plant Mol. Biol. 5,103 (1985); Zhijian et al., Plant Science 108, 219 (1995); Meijer et al., Plant Mol. Bio. 16, 807 (1991)); streptomycin (Jones et al., Mol. Gen. Genet. 210, 86 (1987)); spectinomycin (Bretagne-Sagnard et al., Transgenic Res. 5,131 (1996)); bleomycin (Hille et al., Plant Mol. Biol. 7, 171 (1986)); sulfonamide (Guerineau et al., Plant Mol. Bio. 15,127 (1990); bromoxynil (Stalker et al., Science 242, 419 (1988)); 2,4-D (Streber et al., Bio/Technology 7, 811 (1989)); phosphinothricin (DeBlock et al., EMBO J. 6, 2513 (1987)); spectinomycin (Bretagne-Sagnard and Chupeau, Transgenic Research 5,131 (1996)).

Screenable markers are also used for plant cell transformation, including color markers such as genes encoding β-glucuronidase (gus) or anthocyanin production, or fluorescent markers such as genes encoding luciferase or green fluorescence protein (GFP).

The foregoing list of selectable and screenable marker genes is not meant to be limiting. Any selectable marker gene can be used in the present invention.

An expression vector is a DNA molecule comprising a gene that is expressed in a host cell. Typically, gene expression is placed under the control of certain regulatory regions, including constitutive or inducible promoters, tissue-specific regulatory regions, and enhancers. Such a gene is said to be operably linked to the regulatory regions. An exogenous gene refers in the present description to a gene that is introduced into an organism either from a foreign species, or, if from the same species is substantially modified from its native form in composition and/or genomic locus by deliberate human invention. For example, any gene, even a structural gene normally found in the host plant, is considered to be an exogenous gene, if the gene is reintroduced into the organism.

One of skill in the art will be able to select an appropriate vector for introducing the heterologous nucleic acid sequence in a relatively intact state. Thus, any vector which will produce a plant carrying the introduced DNA sequence should be sufficient. Even a naked piece of DNA would be expected to be able to confer the properties of this invention, though at low efficiency. The selection of the vector, or whether to use a vector, is typically guided by the method of transformation selected.

An endogenous gene refers in the present description to a gene that is in its native form and has not been modified in composition or genomic locus.

A transgenic plant is a plant comprising a DNA region or modification to DNA introduced as a result of the process of transformation.

The term introduced in the context of inserting a nucleic acid into a cell, means transfection or transformation or transduction and includes reference to the incorporation of a nucleic acid into a eukaryotic or prokaryotic cell where the nucleic acid may be incorporated into the genome of the cell (e.g., chromosome, plasmid, plastid or mitochondrial DNA), converted into an autonomous replicon, or transiently expressed (e.g., transfected mRNA).

In eukaryotes, RNA polymerase II catalyzes the transcription of a structural gene to produce mRNA. A DNA molecule can be designed to contain a transcriptional template in which the RNA transcript has a sequence that is complementary to that of a specific mRNA.

Monocots are a large group of flowering plants, having an embryo with one cotyledon, parts of the flowers usually in threes, leaves with parallel veins and vascular bundles scattered throughout the stem. Examples of monocots include maize, barley, rice, sorghum, wheat, forage grass, and turf grass.

Dicots are a large group of flowering plants, having an embryo with two cotelydons, parts of the flower usually in twos or fives or multiples, leaves with net veins, and vascular bundles in the stem in a ring surrounding the central pith. Examples of dicots are vegetables, feed, and oil crops such as tomato, beans, soybeans, peppers, lettuce, peas, alfalfa, clover, Brassica species (e.g., cabbage, broccoli, cauliflower, brussel sprouts, rapeseed, and radish), carrot, beets, eggplant, spinach, cucumber, squash, melons, cantaloupe, sunflowers; fiber crops such as cotton; and various ornamentals such as flowers and shrubs; fruit trees such as apple, citrus.

Gymnosperm plants are also part of this invention which include, but are not limited to, coniferous softwood species such as loblolly pine, Douglas-fir, or Norway spruce.

As used herein, the term plant includes reference to whole plants, plant organs (e.g., leaves, stems, roots, etc.), seeds and plant cells and progeny of same. Plant cell, as used herein includes, without limitation, seeds suspension cultures, embryos, meristematic regions, callus tissue, leaves, roots, shoots, gametophytes, sporophytes, pollen, and microspores. The class of plants which can be used in the methods of the invention is generally as broad as the class of higher plants amenable to transformation techniques, including both monocotyledonous and dicotyledonous plants. A particularly preferred plant is turf grass plant.

T0 refers to the initial transgenic shoot or plant recovered from the transformation and cultural protocols whether the plant is maintained in vitro or established in soil. The T1 generation are those plants resulting from seed recovered from, most commonly, self pollinated T0 plants, or from seed obtained by crosses with other lines where the T0 candidate is either the male or female parent. The T2 generation is the material obtained from T1 selfings or crosses.

The term oxidase as used in this application refers to an enzyme capable of generating hydrogen peroxide or any reactive oxygen species.

A pathogen refers to any organism responsible for disease and/or damage to a plant. For the present invention, pests include but are not limited to insects, fungi, bacteria, nematodes, viruses or viroids, parasitic weeds, and the like.

The term “disease or insect” or “pathogen” or “insect” resistance refers to the ability to maintain a desirable phenotype upon exposure to infection, relative to a nontransgenic plant. The level of resistance can be determined by comparing the physical characteristics of the invention plant to nontransgenic plants that either have or have not been exposed to infection or insect infestation. Exemplary physical characteristics to observe include an increase in population of plants that have the ability to survive pathogen challenge, delayed lesion development, reduced lesion size, and the like. The term “disease” refers to a pathogen challenge caused any agent known to cause symptoms of infection in plants, including, but not limited to bacteria, nematodes, viruses, mycoplasmas, and fungi.

Stress refers to any force that can hurt or damage a plant. Examples of stress are pathogen attack, invasion by a parasitic weed, environmental stress such as heat, cold or drought, or mechanical damage. A stress resistant plant is one that is capable of surviving exposure to a stress. For example, a sunflower plant expressing oxalate oxidase is able to inhibit the establishment of pathogens, such as Sclerotinia sclerotiorum.

For the purposes of the present invention, a plant that is tolerant to a pathogen or other stress is one that is able to withstand a pathogen attack or stressful conditions better than the wild type plant, but will usually succumb to infection and/or die under conditions other than very light disease or stress pressure. A resistant plant is a plant having the ability to exclude or overcome the growth or effects of a pathogen or stress except under extremely high disease or stress pressure. An immune plant is one capable of complete disease resistance, with no reaction of plant tissue to a potential pathogen.

The methods of this invention and the transgenic plants produced, as described in the present application can be used over a broad range of plant types, including species, but are not limited to, from the genera Cucurbita, Rosa, Vitis, Juglans, Fragaria, Lotus, Medicago, Onobrychis, Trifolium, Trigonella, Vigna, Citrus, cotton, Linum, Geranium, Manihot, Daucus, Arabidopsis, Brassica, Raphanus, Sinapis, Atropa, Capsicum, Datura, Hyoscyamus, Lycopersicon, Nicotiana, Solanum, Petunia, Digitalis, Majorana, Ciahorium, Helianthus, Lactuca, Bromus, Asparagus, Antirrhinum, Heterocallis, Nemesis, Pelargonium, Panieum, Pennisetum, Ranunculus, Senecio, Salpiglossis, Cucumis, Browaalia, Glycine, Pisum, Phaseolus, Lolium, Oryza, Zea, Avena, Hordeum, Secale, Triticum, Sorghum, Picea, Caco, and Populus. Pathogens

As noted earlier, the transgenic plants of this the invention can be utilized to protect plants from insect, disease, and parasitic weed pests. For purposes of the present invention, pests include but are not limited to insects, pathogens including fungi, bacteria, nematodes, viruses or viroids, parasitic weeds, and the like. Insect pests include insects selected from the orders Coleoptera, Diptera, Hymenoptera, Lepidoptera, Mallophaga, Homoptera, Hemiptera, Orthoptera, Thysanoptera, Dermaptera, Isoptera, Anoplura, Siphonaptera, Trichoptera, etc., particularly Coleoptera and Lepidoptera. Insect pests of the invention for the major crops include: Maize: Ostrinia nubilalis, European corn borer; Agrotis ipsilon, black cutworm; Helicoverpa zea, corn earworm; Spodoptera frugiperda, fall armyworm; Diatraea grandiosella, southwestern corn borer; Elasmopalpus lignosellus, lesser cornstalk borer; Diatraea saccharalis, sugarcane borer; Diabrotica virgifera, western corn rootworm; Diabrotica longicornis barberi, northern corn rootworm; Diabrotica undecimpunctata howardi, southern corn rootworm; Melanotus spp., wireworms; Cyclocephala borealis, northern masked chafer (white grub); Cyclocephala immaculata, southern masked chafer (white grub); Popillia japonica, Japanese beetle; Chaetocnema pulicaria, corn leaf beetle; Sphenophorus maidis, maize billbug; Rhopalosiphum maidis, corn leaf aphid; Anuraphis maidiradicis, corn root aphid; Blissus leucopterus, chinch bug; Melanoplus femurrubrum, redlegged grasshopper; Melanoplus sanguinipes, migratory grasshopper, Hylemya platura, seedcorn maggot; Agromyza parvicornis, corn bloth leafminer; Anaphothrips obscurus, grass thrips; Solenopsis milesta, thief ant; Tetranychus urticae, twospotted spider mite; Sorghum: Chilo partellus, sorghum borer; Spodoptera frugiperda, fall armyworm; Helicoverpa zea, corn earworm; Elasmopalpus lignosellus, lesser cornstalk borer; Feltia subterranea, granulate cutworm; Phyllophaga crinita, white grub; Eleodes, Conoderus, and Aeolus spp., wireworms; Oulema melanopus, cereal leaf beetle; Chaetocnema pulicaria, corn flea beetle; Sphenophorus maidis, maize billbug; Rhopalosiphum maidis; corn leaf aphid; Sipha flava, yellow sugarcane aphid; Blissus leucopterus; chinch bug; Contarinia sorghicola, sorghum midge; Tetranychus cinnabarinus, carmine spider mite; Tetranychus urticae, twospotted spider mite; Wheat: Pseudaletia unipunctata, army worm; Spodoptera frugiperda, fall armyworm; Elasmopalpus lignosellus, lesser cornstalk borer; Agrotis orthogonia, pale western cutworm; Elasmopalpus lignosellus, lesser cornstalk borer; Oulema melanopus, cereal leaf beetle; Hypera punctata, clover leaf weevil; Diabrotica undecimpunctata howardi, southern corn rootworm; Russian wheat aphid; Schizaphis graminum, greenbug; Macrosiphum avenae, English grain aphid; Melanoplus femurrubrum, redlegged grasshopper; Melanoplus differentialis, differential grasshopper; Melanoplus sanguinipes, migratory grasshopper; Mayetiola destructor, Hessian fly; Sitodiplosis mosellana, wheat midge; Meromyza americana, wheat stem maggot; Hylemya coarctata, wheat bulb fly; Frankliniella fusca, tobacco thrips; Cephus cinctus, wheat stem sawfly; Aceria tulipae, wheat curl mite; Sunflower: Suleima helianthana, sunflower bud moth; Homoeosoma electellum, sunflower moth; Zygogramma exclamationis, sunflower beetle; Bothyrus gibbosus, carrot beetle; Neolasioptera murtfeldtiana, sunflower seed midge; Cotton: Heliothis virescens, cotton boll worm; Helicoverpa zea, cotton bollworm; Spodoptera exigua, beet armyworm; Pectinophora gossypiella, pink bollworm; Anthonomus grandis, boll weevil; Aphis gossypii, cotton aphid; Pseudatomoscelis seriatus, cotton fleahopper; Trialeurodes abutilonea, bandedwinged whitefly; Lygus lineolaris, tarnished plant bug; Melanoplus femurrubrum, redlegged grasshopper; Melanoplus differentialis, differential grasshopper; Thrips tabaci, onion thrips; Frankliniella fusca, tobacco thrips; Tetranychus urticae, twospotted spider mite; Rice: Diatraea saccharalis, sugarcane borer; Spodoptera frugiperda, fall armyworm; Helicoverpa zea, corn earworm; Colaspis brunnea, grape colaspis; Lissorhoptrus oryzophilus, rice water weevil; Sitophilus oryzae, rice weevil; Nephotettix nigropictus, rice leafhopper; Blissus leucopterus, chinch bug; Acrosternum hilare, green stink bug; Soybean: Pseudoplusia includens, soybean looper; Anticarsia gemmatalis, velvetbean caterpillar; Plathypena scabra, green cloverworm; Ostrinia nubilalis, European corn borer; Agrotis ipsilon, black cutworm; Spodoptera exigua, beet armyworm; Heliothis virescens, cotton boll worm; Helicoverpa zea, cotton bollworm; Epilachna varivestis, Mexican bean beetle; Myzus persicae, green stink bug; Melanoplus femurrubrum, redlegged grasshopper; Melanoplus differentialis, differential grasshopper; Hylemya platura, seedcorn maggot; Sericothrips variabilis, soybean thrips; Thrips tabaci, onion thrips; Tetranychus turkestani, strawberry spider mite; Tetranychus urticae, twospotted spider mite; Barley: Ostrinia nubilalis, European corn borer; Agrotis ipsilon, black cutworm; Schizaphis graminum, greenbug; Blissus leucopterus leucopterus, chinch bug; Acrostemum hilare, green stink bug; Euschistus servus, brown stink bug; Jylemya platura, seedcorn maggot; Mayetiola destructo, Hessian fly; Petrobia latens, brown seedcorn maggot; Mayetiola destructor, Hessian fly; Petrobia latens, brown seedcorn maggot; Mayetiola destructor, Hessian fly; Petrobia latens, brown wheat mite; Oil Seed Rape: Vrevicoryne brassicae, cabbage aphid.

Generally Viruses include tobacco or cucumber mosaic virus, ringspot virus, necrosis virus, maize dwarf mosaic virus, etc. specific viral, fungal and bacterial pathogens for the major crops include: Soybeans: Phytophthora megasperma fsp. Glycinea, Macrophomina phaseolina, Rhizoctonia solani, Sclerotinia sclerotiorum, Fusarium oxysporum, Diaporthe phaseolorum var. sojae (Phomopsis sojae), Diaporthe phaseolorum var. caulivora, Sclerotium rolfsii, Cercospora kikuchii, Cercospora sojina, Peronospora manshurica, Colletotrichum dematium (Colletotrichum truncatum), Corynespora cassiicola, Septoria glycines, Phyllosticta sojicola, Alternaria alternata, Pseudomonas syringae p.v. glycinea, Xanthomonas campestris p.v. phaseoli, Microsphaera diffusa, Fusarium semitectum, Phialophora gregata, Soybean mosaic virus, Glomerella glycines, Tobacco Ring spot virus, Tobacco Streak virus, Phakopsora pachyrhizi, Pythium aphanidermatum, Pythium ultimum, Pythium debaryanum, Tomato spotted wilt virus, Heterodera glycines Fusarium solani; Canola: Albugo candida, Alternaria brassicae, Leptosphaeria maculans, Rhizoctonia solani, Sclerotinia sclerotiorum, Mycosphaerella brassiccola, Pythium ultimum, Peronospora parasitica, Fusarium roseum, Alternaria alternata; Alfalfa: Clavibater michiganensis subsp. Insidiosum, Pythium ultimum, Pythium irregulare, Pythium splendens, Pythium debaryanum, Pythium aphanidermatum, Phytophthora megasperma, Peronospora trifoliorum, Phoma medicaginis var. medicaginis, Cercospora medicaginis, Pseudopeziza medicaginis, Leptotrochila medicaginis, Fusarium oxysporum, Rhizoctonia solani, Uromyces striatus, Colletotrichum trifolii race 1 and race 2, Leptosphaerulina briosiana, Stemphylium botryosum, Stagonospora meliloti, Sclerotinia trifoliorum, Alfalfa Mosaic Virus, Verticillium albo-atrum, Xanthomonas campestris p.v. alfalfae, Aphanomyces euteiches, Stemphylium herbarum, Stemphylium alfalfae; Wheat: Pseudomonas syringae p.v. atrofaciens, Urocystis agropyri, Xanthomonas campestris p.v. translucens, Pseudomonas syringae p.v. syringae, Alternaria alternata, Cladosporium herbarum, Fusarium graminearum, Fusarium avenaceum, Fusarium culmorum, Ustilago tritici, Ascochyta tritici, Cephalosporium gramineum, Colletotrichum graminicola, Erysiphe graminis f.sp. tritici, Puccinia graminis f.sp. tritici, Puccinia recondita f.sp. tritici, Puccinia striiformis, Pyrenophora tritici-repentis, Septoria nodorum, Septoria tritici, Septoria avenae, Pseudocercosporella herptotrichoides, Rhizoctonia solani, Rhizoctonia cerealis, Gaeumannomyces graminis var. tritici, Pythium aphanidermatum, Pythium arrhenomanes, Pythium ultimum, Bipolaris sorokiniana, Barley Yellow Dwarf Virus, Brome Mosaic Virus, Soil Borne Wheat Mosaic Virus, Wheat Streak Mosaic Virus, Wheat Spindle Streak Virus, American Wheat Striate Virus, Claviceps purpurea, Tilletia tritici, Tilletia laevis, Ustilago tritici, Tilletia indica, Rhizoctonia solani, Pythium arrhenomanes, Pythium graminicola, Pythium aphanidermatum, High Plains Virus, European wheat striate virus; Sunflower: Plasmophora halstedii, Sclerotinia sclerotiorum, Aster Yellows, Septoria helianthi, Phomopsis helianthi, Alternaria helianthi, Alternaria zinniae, Botrytis cinerea, Phoma macdonaldii, Macrophomina phaseolina, Erysiphe cichoracearum, Rhizopus oryzae, Rhizopus arrhizus, Rhizopus stolonifer, Puccinia helianthi, Verticillium dahlia, Erwinia carotovora pv. carotovora, Cephalosporium acremonium, Phytophthora cryptogea, Albugo tragopogonis; Maize: Fusarium moniliforme var. subglutinans, Erwinia stewartii, Fusarium moniliforme, Gibberella zeae (Fusarium graminearum), Stenocarpella maydis (Diplodia maydis), Pythium irregulare, pythium debaryanum, Pythium graminicola, Pythium splendens, Pythium ultimum, Pythium aphanidermatum, Aspergillus flavus, Bipolaris maydis O, T Cochliobolus heterostrophus), Helminthosporium carbonum I, II & III (Cochliobolus carbonum), Exserohilum turcicum I, II & III, Helminthosporium pedicellatum, Physoderma maydis, Phyllosticta maydis, Kabatiella zea, Colletotrichum graminicola, Cercospora zeae-maydis, Cercospora sorghi, Ustilago maydis, Puccinia sorghi, Puccinia polysora, Macrophomina phaseolina, Penicillium oxalicum, Nigrospora oryzae, Cladosporium herbarum, Curvularia lunata, Curvularia inaequalis, Curvularia pallescens, Clavibacter michiganense subsp. nebraskense, Trichoderma viride, Maize Dwarf Mosaic Virus A & B, Wheat Streak Mosaic Virus, Maize Chlorotic Dwarf Virus, Claviceps sorghi, Pseudonomas avenae, Erwinia chrysanthemi pv. Zea, Erwinia carotovora, Corn stunt spiroplasma, Diplodia macrospora, Sclerophthora macrospora, Peronosclerospora sorghi, Peronosclerospora philippinensis, Peronosclerospora maydis, Peronosclerospora sacchari, Spacelotheca reiliana, Physopella zeae, Cephalosporium maydis, Cephalosporium acremonium, Maize chlorotic mottle virus, High plains virus, Maize mosaic virus, Maize rayado fino virus, Maize streak virus, Maize stripe virus, Maize rough dwarf virus; Sorghum: Exserohilum turcicum, Colletotrichum graminicola (Glomerella graminicola), Cercospora sorghi, Gloeocercospora sorghi, Ascochyta sorghi, Pseudomonas syringae p.v. syringae, Xanthomonas campestris p.v. holcicola, Pseudomonas andropogonis, Puccinia purpurea, Macrophomina phaseolina, Periconia circinata, Fusarium moniliforme, Alternaria alternate, Bipolaris sorghicola, Helminthosporium sorghicola, Curvularia lunata, Phoma insidiosa, Pseudomonas avenae (Pseudomonas alboprecipitans), Ramulispora sorghi, Ramulispora sorghicola, Phyllachara sacchari, Sporisorium relianum (Sphacelotheca reliana), Sphacelotheca cruenta, Sporisorium sorghi, Sugarcane mosaic H, Maize Dwarf Mosaic Virus A & B, Claviceps sorghi, Rhizoctonia solani, Acremonium strictum, Sclerophthona macrospora, Peronosclerospora sorghi, Peronosclerospora philippinensis, Sclerospora graminicola, Fusarium graminearum, Fusarium Oxysporum, Pythium arrhenomanes, Pythium graminicola, etc.

Generally parasitic weeds include the parasitic flowering plants Orobanche spp. (Broomrape), the mistletoes (Lorranthaceae: genera Arceuthobrium, Viscum, and Phoradendron, dodder (Cuscuta spp.), and Striga spp. (Witchweeds). Parasitic weeds of the present invention include, but are not limited to, Sunflower and Canola: Orobanche aegyptiaca, Orabanche cumana, Tomato and Potato: Orobanche aegyptiaca, Orobanche ramosa, Orobanche cernua, etc.

In order to express the genes, homologues or functional fragments of this invention, a promoter must be operably linked to that gene, homologues or functional fragment. Many different constitutive promoters can be utilized in the instant invention to express the gene of interest. Examples include promoters from plant viruses such as the 35S promoter from cauliflower mosaic virus (CaMV), as described in Odell, et al., Nature, 313: 810-812 (1985), and hereby incorporated by reference, and promoters from genes such as rice actin (McElroy, et al., Plant Cell, 163-171 (1990)); ubiquitin (Christensen, et al., Plant Mol. Biol., 12: 619-632 (1992); and Christensen, et al., Plant Mol. Biol., 18: 675-689 (1992)); pEMU (Last, et al., Theor. Appl. Genet., 81: 581-588 (1991)); MAS (Velten, et al., EMBO J., 3: 2723-2730 (1984)); maize H3 histone (Lepetit, et al., Mol. Gen. Genet., 231: 276-285 (1992); and Atanassvoa, et al., Plant Journal, 2(3): 291-300 (1992)), the 1′- or 2′-promoter derived from T-DNA of Agrobacterium tumefaciens, the Smas promoter, the cinnamyl alcohol dehydrogenase promoter (U.S. Pat. No. 5,683,439), the Nos promoter, the rubisco promoter, the GRP1-8 promoter, ALS promoter, as described in published PCT application WO 96/30530, a synthetic promoter, such as, Rsyn7, SCP and UCP promoters as described in U.S. patent application Ser. No. 09/028,819, filed Feb. 24, 1998 and herein incorporated by reference, and other transcription initiation regions from various plant genes known to those of skill. In the present invention, an expression vector comprises a constitutive promoter operationally linked to a nucleotide sequence encoding for a hydrogen peroxide/reactive oxygen species producing gene. The expression vector and an accompanying, selectable marker gene under the direction of a plant-expressible constitutive promoter are introduced into plant cells, selective agent-resistant cells or tissues are recovered, resistant plants are regenerated and T0 candidates are screened for enzyme activity in leaf samples. T0 candidates can also be obtained without the use of a selectable marker. In this instance, the expression vector is introduced into plant cells without an accompanying selectable marker gene and transformed tissues are identified and plants screened based on enzyme activity alone.

Additional regulatory elements that may be a nucleic acid sequence for expression in plant cells include terminators, polyadenylation sequences, and nucleic acid sequences encoding signal peptides that permit localization within a plant cell or secretion of the protein from the cell. Such regulatory elements and methods for adding or exchanging these elements with the regulatory elements of the gene are known, and include, but are not limited to, 3′ termination and/or polyadenylation regions such as those of the Agrobacterium tumefaciens nopaline synthase (nos) gene (Bevan, et al., Nucl. Acids Res., 12: 369-385 (1983)); the potato proteinase inhibitor II (PINII) gene (Keil, et al., Nucl. Acids Res., 14: 5641-5650 (1986) and hereby incorporated by reference); and An, et al., Plant Cell, 1: 115-122 (1989)); and the CaMV 19S gene (Mogen, et al., Plant Cell, 2: 1261-1272 (1990)).

Plant signal sequences, including, but not limited to, signal-peptide encoding DNA/RNA sequences which target proteins to the extracellular matrix of the plant cell (Dratewka-Kos, et al., J. Biol. Chem., 264: 4896-4900 (1989)) and the Nicotiana plumbaginifolia extension gene (DeLoose, et al., Gene, 99: 95-100 (1991)), or signal peptides which target proteins to the vacuole like the sweet potato sporamin gene (Matsuka, et al., Proc. Nat'l Acad. Sci. (USA), 88: 834 (1991)) and the barley lectin gene (Wilkins, et al., Plant Cell, 2: 301-313 (1990)), or signals which cause proteins to be secreted such as that of PRIb (Lind, et al., Plant Mol. Biol., 18: 47-53 (1992)), or those which target proteins to the plastids such as that of rapeseed enoyl-Acp reductase (Verwaert, et al., Plant Mol. Biol., 26: 189-202 (1994)) are useful in the invention. An especially useful signal sequence for this invention is signal sequence isolated from the oxalate oxidase gene. (Lane, et al., J. Biol. Chem., 266(16): 10461-10469 (1991)) Gene

### Transformation Methods

Numerous methods for introducing foreign genes into plants are known and can be used to insert a gene into a plant host, including biological and physical plant transformation protocols. See, for example, Miki et al., (1993) “Procedure for Introducing Foreign DNA into Plants”, In: Methods in Plant Molecular Biology and Biotechnology, Glick and Thompson, eds., CRC Press, Inc., Boca Raton, pages 67-88. The methods chosen vary with the host plant, and include chemical transfection methods such as calcium phosphate, microorganism-mediated gene transfer such as Agrobacterium (Horsch, et al., Science, 227: 1229-31 (1985)), electroporation, micro-injection, and biolistic bombardment.

Expression cassettes and vectors and in vitro culture methods for plant cell or tissue transformation and regeneration of plants are known and available. See, for example, Gruber, et al., (1993) “Vectors for Plant Transformation” In: Methods in Plant Molecular Biology and Biotechnology, Glick and Thompson, eds. CRC Press, Inc., Boca Raton, pages 89-119. Agrobacterium-mediated Transformation The most widely utilized method for introducing an expression vector into plants is based on the natural transformation system of Agrobacterium. A. tumefaciens and A. rhizogenes are plant pathogenic soil bacteria which genetically transform plant cells. The Ti and Ri plasmids of A. tumefaciens and A. rhizogenes, respectfully, carry genes responsible for genetic transformation of plants. See, for example, Kado, Crit. Rev. Plant Sci., 10:1-32 (1991). Descriptions of the Agrobacterium vector systems and methods for Agrobacterium-mediated gene transfer are provided in Gruber et al., supra; and Moloney, et al, Plant Cell Reports, 8: 238-242 (1989). Direct Gene Transfer Despite the fact that the host range for Agrobacterium-mediated transformation is broad, some major cereal crop species and gymnosperms have generally been recalcitrant to this mode of gene transfer, even though some success has recently been achieved in rice (Hiei et al., The Plant Journal, 6: 271-282 (1994)) and maize (Ishida, et al., Nature Biotech., 14: 754-750 (1996)). Several methods of plant transformation, collectively referred to as direct gene transfer, have been developed as an alternative to Agrobacterium-mediated transformation.

A generally applicable method of plant transformation is microprojectile-mediated transformation, where DNA is carried on the surface of microprojectiles measuring about 1 to 4 μm. The expression vector is introduced into plant tissues with a biolistic device that accelerates the microprojectiles to speeds of 300 to 600 m/s which is sufficient to penetrate the plant cell walls and membranes. (Sanford, et al., Part. Sci. Technol., 5: 27-37 (1987); Sanford, Trends Biotech, 6: 299-302 (1988); Sanford, Physiol. Plant, 79: 206-209 (1990); Klein, et al., Biotechnology, 10: 286-291 (1992)).

Another method for physical delivery of DNA to plants is sonication of target cells as described in Zang, et al., BioTechnology, 9: 996-996 (1991). Alternatively, liposome or spheroplast fusions have been used to introduce expression vectors into plants. See, for example, Deshayes, et al., EMBO J., 4: 2731-2737 (1985); and Christou, et al., Proc. Nat'l. Acad. Sci. (USA), 84: 3962-3966 (1987). Direct uptake of DNA into protoplasts using CaCl₂ precipitation, polyvinyl alcohol or poly-L-ornithine have also been reported. See, for example, Hain, et al., Mol. Gen. Genet., 199: 161 (1985); and Draper, et al., Plant Cell Physiol., 23: 451-458 (1982).

Electroporation of protoplasts and whole cells and tissues has also been described. See, for example, Donn, et al., (1990) In: Abstracts of the VIIth Int;l. Congress on Plant Cell and Tissue Culture IAPTC, A2-38, page 53; D'Halluin et al., Plant Cell, 4: 1495-1505 (1992); and Spencer et al., Plant Mol. Biol., 24: 51-61 (1994). Particle Wounding/Agrobacterium Delivery

Another useful basic transformation protocol involves a combination of wounding by particle bombardment, followed by use of Agrobacterium for DNA delivery, as described by Bidney, et al., Plant Mol. Biol., 18: 301-31 (1992). Useful plasmids for plant transformation include Bin 19. See Bevan, Nucleic Acids Research, 12: 8711-8721 (1984), and hereby incorporated by reference.

In general, the intact meristem transformation method involves imbibing seed for 24 hours in the dark, removing the cotyledons and root radical, followed by culturing of the meristem explants. Twenty-four hours later, the primary leaves are removed to expose the apical meristem. The explants are placed apical dome side up and bombarded, e.g., twice with particles, followed by co-cultivation with Agrobacterium. To start the co-cultivation for intact meristems, Agrobacterium is placed on the meristem. After about a 3-day co-cultivation period the meristems are transferred to culture medium with cefotaxime plus kanamycin for the NPTII selection.

The split meristem method involves imbibing seed, breaking of the cotyledons to produce a clean fracture at the plane of the embryonic axis, excising the root tip and then bisecting the explants longitudinally between the primordial leaves. The two halves are placed cut surface up on the medium then bombarded twice with particles, followed by co-cultivation with Agrobacterium. For split meristems, after bombardment, the meristems are placed in an Agrobacterium suspension for 30 minutes. They are then removed from the suspension onto solid culture medium for three day co-cultivation. After this period, the meristems are transferred to fresh medium with cefotaxime plus kanamycin for selection. Transfer by Plant Breeding

Alternatively, once a single transformed plant has been obtained by the foregoing recombinant DNA method, conventional plant breeding methods can be used to transfer the gene and associated regulatory sequences via crossing and backcrossing. Such intermediate methods will comprise the further steps of: (1) sexually crossing the disease-resistant plant with a plant from the disease susceptible taxon; (2) recovering reproductive material from the progeny of the cross; and (3) growing disease-resistant plants from the reproductive material. Where desirable or necessary, the agronomic characteristics of the susceptible taxon can be substantially preserved by expanding this method to include the further steps of repetitively: (1) backcrossing the disease-resistant progeny with disease-susceptible plants from the susceptible taxon; and (2) selecting for expression of a hydrogen peroxide producing enzyme activity (or an associated marker gene) among the progeny of the backcross, until the desired percentage of the characteristics of the susceptible taxon are present in the progeny along with the gene or genes imparting oxalic acid degrading and/or hydrogen peroxide enzyme activity. The same methodology can be used to introduce the genes that are part of this invention.

By the term “taxon” herein is meant a unit of botanical classification. It thus includes, genus, species, cultivars, varieties, variants and other minor taxonomic groups which lack a consistent nomenclature.

The combination of more that one of the genes of this invention in a plant having a pathogen tolerant genetic background yields unexpectedly superior disease resistance compared to the expression of any of these genes in a non-tolerant background. Only in combination, does an immune or near immune plant result.

The presence of the heterologous nucleic acid or “transgene(s)” in the seeds of the transformed plant or in the regenerated plants produced from those seeds, a variety of assays may be performed. Such assays include, for example, molecular biological assays, such as Southern and Northern blotting and PCR; biochemical assays, such as detecting the presence of a protein product, by immunological means (ELISAs and Western blots) or by enzymatic function; and by plant part assays, such as leaf or root assays. While Southern blotting and PCR may be used to detect the gene(s) in question, they do not provide information as to whether the gene is being expressed. Expression of the heterologous gene may be evaluated by specifically identifying the protein products of the introduced genes or evaluating the phenotypic changes brought about by their expression. Assays for the production and identification of specific proteins may make use of physical-chemical, structural, functional, or other properties of the proteins. Unique physical-chemical or structural properties allow the proteins to be separated and identified by electrophoretic procedures, such as native or denaturing gel electrophoresis or isoelectric focusing, or by chromatographic techniques such as ion exchange or gel exclusion chromatography. The unique structures of individual proteins offer opportunities for use of specific antibodies to detect their presence in formats such as an ELISA assay. Combinations of approaches may be employed with even greater specificity such as Western blotting in which antibodies are used to locate individual gene products that have been separated by electrophoretic techniques. Additional techniques may be employed to absolutely confirm the identity of the product of interest such as evaluation by amino acid sequencing following purification. Although these techniques are among the most commonly employed, other procedures are known in the art and may be additionally used.

Transgenic plants comprising the heterologous nucleic acid of the present invention (e.g., comprising a heterologous nucleic acid of the present invention, or a transformed cell of the present invention), as well as the seeds and progeny produced by the transgenic plants are also an aspect of the present invention. Procedures for cultivating transformed cells to useful cultivars are known to those skilled in the art. Techniques are known for the in vitro culture of plant tissue, and in a number of cases, for regeneration into whole plants. A further aspect of the invention is transgenic plant tissue, plants or seeds containing the nucleic acids described above. In a preferred embodiment, transformed explants produced using the present invention are not chimeric, or only a small proportion of transformed explants is chimeric.

Plants produced by the methods of the present invention may be screened for successful transformation by standard methods described above. Seeds and progeny plants of regenerated plants of the present invention may be continuously screened and selected for the continued presence of the transgenic and integrated nucleic acid sequence in order to develop improved plant and seed lines, which are an another aspect of the present invention. Desirable transgenic nucleic acid sequences may thus be moved (i.e., introgressed or inbred) into other genetic lines such as certain elite or commercially valuable lines or varieties. Methods of introgressing desirable nucleic acid sequences into genetic plant lines may be carried out by a variety of techniques known in the art, including by classical breeding, protoplast fusion, nuclear transfer and chromosome transfer. Breeding approaches and techniques are known in the art, and are set forth in, for example, J. R. Welsh, Fundamentals of Plant Genetics and Breeding (John Wiley and Sons, New York, (1981)); Crop Breeding (D. R. Wood, ed., American Society of Agronomy, Madison, Wis., (1983)); O. Mayo, The Theory of Plant Breeding, Second Edition (Ciarendon Press, Oxford, England (1987)); and Wricke and Weber, Quantitative Genetics and Selection Plant Breeding (Walter de Gruyter and Co., Berlin (1986)). Using these and other techniques in the art, transgenic plants and inbred lines obtained according to the present invention may be used to produce commercially valuable hybrid plants and crops (e.g., hybrid squash and sugarbeets), which hybrids are also an aspect of the present invention.

The term “BAS 1 polypeptide” as used herein means the BAS 1 polypeptide having the amino acid sequence of SEQ ID NO: 2, as well as functional fragments thereof, along with other homologous plant cytochrome P450s, such as CYP72A from Catharanthus roseus (Madagascar periwinkle), which has about 42% sequence identity with BAS1 at the amino acid level and the CYP72 chibi2 from Arabidopsis.

The invention includes functional BAS1 polypeptide, and functional fragments thereof. As used herein, the term “functional polypeptide” refers to a polypeptide which possesses biological function or activity which is identified through a functional assay (e.g., brassinolide activity) and which is associated with a particular biologic, morphologic, or phenotypic alteration in the cell. For example, overexpression of BAS1 polypeptide results in modulation of brassinolide activity, characterized by one or more of the following: hypersensitivity to far-red light in a PHYA background and lack of responsiveness in a phyA null background, etiolation with hypocotyls of near wild-type length in dark grown seedlings, and dwarfism with dark-green leaves in adult plants.

The term “functional fragments of BAS 1 polypeptide”, refers to all fragments of BAS1 that retain BAS1 activity, e.g., being a dominant suppressor of phyB-4, being a cytochrome P450, modulating brassinolide activity in plants. Biologically functional fragments, for example, can vary in size from a polypeptide fragment as small as an epitope capable of binding an antibody molecule to a large polypeptide capable of participating in the characteristic induction or programming of phenotypic changes within a cell. Functional fragments of BAS I include antigenic fragments.

For example, BAS1 may modulate brassinolide activity in diverse tissues, or in a tissue specific manner; therefore an assay can be performed to detect BAS 1 brassinolide activity. Inhibitors of BAS 1, such as BAS 1 antisense nucleic acids, could be used to cause loss of function of BAS I resulting in, for example, hypocotyls that are slightly longer than the wild type in dark growth, and have a reduced responsiveness to white, far-red and blue light, compared with wild type plants.

The polypeptides of the invention also include dominant negative forms of the BAS 1 polypeptide that do not have the biological activity of BAS 1. A “dominant negative form” of BAS 1 is a polypeptide that is structurally similar to BAS 1 but does not have wild-type BAS 1 function. For example, a dominant-negative BAS 1 polypeptide may interfere with wild-type BAS 1 function by binding to, or otherwise sequestering, regulating agents, such as upstream or downstream components, that normally interact functionally with the BAS 1 polypeptide. More details are found in U.S. Pat. No. 6,534,313, WO0055302, US20020073446 which are incorporated herein by a reference.

Minor modifications of the BAS 1 primary amino acid sequence may result in proteins that have substantially equivalent activity to the BAS 1 polypeptide described herein in SEQ ID NO: 2 (FIG. 1 in U.S. Pat. No. 6,534,313, WO0055302, US20020073446. The content of these issued patents and published patent applications are herein incorporated in full by a reference. The nucleic acid and amino acid sequences in these patents as well as any reference mentioned in this invention is part of this invention). Such modifications may be deliberate, as by site directed mutagenesis, or may be spontaneous. All of the polypeptides produced by these modifications are included herein, as long as the biological activity of BAS 1 is present, e.g., modification of brassinolide or ecdysteroid synthesis and/or signaling activity is present. Further, deletion of one or more amino acids can also result in a modification of the structure of the resultant molecule without significantly altering its activity. This can lead to the development of a smaller active molecule that could have broader utility. For example, it may be possible to remove amino or carboxy terminal amino acids not required for BAS 1 activity.

BAS 1 polypeptide includes amino acid sequences substantially the same as the sequence set forth in SEQ ID NO: 2 (U.S. Pat. No. 6,534,313, WO0055302, US20020073446). The content of these issued patents and published patent applications are herein incorporated in full by a reference. The invention includes polypeptides having substantially the same sequence of amino acids as the amino acid sequence set forth in

SEQ ID NO: 2, functional fragments thereof, and amino acid sequences that are substantially identical to SEQ ID NO: 2. By “substantially the same” or “substantially identical” is meant a polypeptide or nucleic acid exhibiting at least 80%, preferably 85%, more preferably 90%, and most preferably 95% homology to a reference amino acid or nucleic acid sequence. For polypeptides, the length of comparison sequences will generally be at least 16 amino acids, preferably at least 20 amino acids, more preferably at least 25 amino acids, and most preferably 35 amino acids. For nucleic acids, the length of comparison sequences will generally be at least 50 nucleotides, preferably at least 60 nucleotides, more preferably at least 75 nucleotides, and most preferably 110 nucleotides. BAS 1 homologs can be identified as having a % homology with BAS 1 within these ranges.

Functional fragments include those fragments of BAS 1 that retain the function or activity of BAS 1, such as the ability to modulate brassinolide synthesis or signaling. One of skill in the art can screen for the functionality of a fragment by using the examples provided herein, where full-length BAS 1 is described. It is also envisioned that fragments of BAS 1 that inhibit or promote brassinolide synthesis or signaling can be identified in a similar manner.

By “substantially identical” is also meant an amino acid that has one or more non-conservative substitutions, deletions, or insertions located at positions of the amino acid sequence which do not destroy the function of the protein assayed, (e.g., as described herein). Preferably, such a sequence is at least 85%, more preferably 100% identical at the amino acid level to SEQ ID NO: 2 (U.S. Pat. No. 6,534,313, WO0055302, US20020073446). The content of these issued patents and published patent applications are herein incorporated in full by a reference.

Homology is often measured using sequence analysis software (e.g., Sequence Analysis Software Package of the Genetics Computer Group, University of Wisconsin Biotechnology Center, 1710 University Avenue, Madison, Wis. 53705). Such software matches similar sequences by assigning degrees of homology to various substitutions, deletions, substitutions, and other modifications.

By a “substantially pure polypeptide” is meant a BAS 1 polypeptide that has been separated from components that naturally accompany it. Typically, the polypeptide is substantially pure when it is at least 60%, by weight, free from the proteins and naturally-occurring organic molecules with which it is naturally associated. Preferably, the preparation is at least 75%, more preferably at least 90%, and most preferably at least 99%, by weight, BAS 1 polypeptide. A substantially pure

BAS 1 polypeptide may be obtained, for example, by extraction from a natural source (e.g., a plant cell); by expression of a recombinant nucleic acid encoding a BAS 1 polypeptide; or by chemically synthesizing the protein. Purity can be measured by any appropriate method, e.g., those described in column chromatography, polyacrylamide gel electrophoresis, or HPLC analysis.

A protein is substantially free of naturally associated components when it is separated from those contaminants which accompany it in its natural state. Thus, a protein which is chemically synthesized or produced in a cellular system different from the cell from which it naturally originates will be substantially free from its naturally associated components. Accordingly, substantially pure polypeptides include those derived from eukaryotic organisms, but synthesized in E. coli or other prokaryotes.

The invention provides polynucleotides encoding the BAS 1 protein. These polynucleotides include DNA, cDNA and RNA sequences which encode BAS 1. It is understood that all polynucleotides encoding BAS1 are also included herein, as long as they encode a polypeptide with BAS 1 activity. Such polynucleotides include naturally occurring, synthetic, and intentionally manipulated polynucleotides. For example, BAS1 polynucleotide may be subjected to site-directed mutagenesis. The polynucleotide sequence encoding BAS1 also includes antisense sequences, sequences encoding dominant negative forms of BAS 1, and sequences encoding

BAS 1 fragments or peptides. The polynucleotides of the invention include sequences that are degenerate as a result of the genetic code. There are 20 natural amino acids, most of which are specified by more than one codon. Therefore, all degenerate nucleotide sequences are included in the invention as long as the amino acid sequence of BAS 1 polypeptide encoded by the nucleotide sequence is functionally unchanged.

Specifically disclosed herein is a polynucleotide sequence containing the BAS1 gene. Preferably, the BAS1 nucleotide sequence is SEQ ID NO: 1. (U.S. Pat. No. 6,534,313, WO0055302, US20020073446). The content of these issued patents and published patent applications are herein incorporated in full by a reference. The term “polynucleotide” or “nucleic acid sequence” refers to a polymeric form of nucleotides at least 10 bases in length. By “isolated polynucleotide” or “purified polynucleotide” is meant a polynucleotide that is not immediately contiguous with both of the coding sequences with which it is immediately contiguous (one on the 5′ end and one on the 3′ end) in the naturally occurring genome of the organism from which it is derived.

The term therefore includes, for example, a recombinant DNA which is incorporated into a vector; into an autonomously replicating plasmid or virus; or into the genomic DNA of a prokaryote or eukaryote; or which exists as a separate molecule (e.g. a cDNA) independent of other sequences. The nucleotides of the invention can be ribonucleotides, deoxyribonucleotides, or modified forms of either nucleotide. The term includes single and double forms of DNA. It also includes a recombinant DNA that is part of a hybrid gene encoding additional polypeptide sequence.

The invention also provides an isolated polynucleotide sequence encoding a polypeptide having the amino acid sequence of SEQ ID NO: 2. (U.S. Pat. No. 6,534,313, WO0055302, US20020073446). The content of these issued patents and published patent applications are herein incorporated in full by a reference. The BAS1 transcript contains a single, long open reading frame that encodes an approximately 510-amino acid protein.

The polynucleotide encoding BAS 1 includes the nucleotide sequence in FIG. 1 (SEQ ID NO: 1. (U.S. Pat. No. 6,534,313, WO0055302, US20020073446)) The content of these issued patents and published patent applications are herein incorporated in full by a reference), as well as nucleic acid sequences complementary to that sequence. A complementary sequence may include an antisense nucleotide. When the sequence is RNA, the deoxyribonucleotides A, G, C, and T of FIG. 1 are replaced by ribonucleotides A, G, C, and U, respectively. Also included in the invention are fragments (“probes”) of the above-described nucleic acid sequences that are at least 15 bases in length, which is sufficient length to permit the probe to selectively hybridize to DNA that encodes the protein of FIG. 1 (SEQ ID NO: 2). (U.S. Pat. No. 6,534,313, WO0055302, US20020073446). The content of these issued patents and published patent applications are herein incorporated in full by a reference.

“Selective hybridization” as used herein refers to hybridization under moderately stringent or highly stringent physiological conditions (See, for example, the techniques described in Maniatis et al., Molecular Cloning A Laboratory Manual, Cold Spring Harbor Laboratory, N.Y., 1989, incorporated herein by reference), which distinguishes related from unrelated BAS1 nucleotide sequences.

Specifically disclosed herein is a cDNA sequence for the BAS1 gene. FIG. 1 shows the complete cDNA and deduced protein sequences (SEQ ID NO: 1 and 2, respectively). (U.S. Pat. No. 6,534,313, WO0055302, US20020073446). The content of these issued patents and published patent applications are herein incorporated in full by a reference.

A “substantially identical” nucleic acid sequence codes for a substantially identical amino acid sequence as defined above.

In nucleic acid hybridization reactions, the conditions used to achieve a particular level of stringency will vary, depending on the nature of the nucleic acids being hybridized. For example, the length, degree of complementarity, nucleotide sequence composition (e.g., GC v. AT content), and nucleic acid type (e.g., RNA v.DNA) of the hybridizing regions of the nucleic acids can be considered in selecting hybridization conditions. An additional consideration is whether one of the nucleic acids is immobilized, for example, on a filter. An example of progressively higher stringency conditions is as follows: 2×SSC/0.1% SDS at about room temperature (hybridization conditions); 0.2×SSC/0.1% SDS at about room temperature (low stringency conditions); 0.2×SSC/0.1% SDS at about 42 C (moderate stringency conditions); and 0.1×SSC at about 68 C (high stringency conditions). Washing can be carried out using only one of these conditions, e.g., high stringency conditions, or each of the conditions can be used, e.g., for 10-15 minutes each, in the order listed above, repeating any or all of the steps listed. However, as mentioned above, optimal conditions will vary, depending on the particular hybridization reaction involved, and can be determined empirically.

A polynucleotide sequence encoding a BAS 1 polypeptide of the invention includes nucleotide sequences encoding the disclosed sequence (e.g., SEQ ID NO: 2) and conservative variations thereof. The term “conservative variation” as used herein denotes the replacement of an amino acid residue by another, biologically similar residue. Examples of conservative variations include the substitution of one hydrophobic residue, such as isoleucine, valine, leucine or methionine, for another, or the substitution of one polar residue for another, such as the substitution of arginine for lysine, glutamic for aspartic acid, or glutamine for asparagine, and the like. The term “conservative variation” also includes the use of a substituted amino acid in place of an unsubstituted parent amino acid provided that antibodies raised to the substituted polypeptide also immunoreact with the unsubstituted polypeptide.

DNA sequences encoding BAS 1 can be expressed in vitro by DNA transfer into a suitable host cell. “Host cells” are cells in which a vector can be propagated and its DNA expressed. The cell may be prokaryotic or eukaryotic. The term also includes any progeny of the subject host cell. It is understood that all progeny may not be identical to the parental cell since there may be mutations that occur during replication. However, such progeny are included when the term “host cell” is used.

As used herein, the term “contacting” refers to any means of introducing the vector (s) into the plant cell, including chemical and physical means as described above. Preferably, contacting refers to introducing the nucleic acid or vector into plant cells (including an explant, a meristem or a seed), via Agrobacterium tumefaciens transformed with the heterologous nucleic acid as described above.

The term “genetic modification” as used herein refers to the introduction of one or more heterologous nucleic acid sequences into one or more plant cells, to provide sexually competent, viable plants. The term “genetically modified” as used herein refers to a plant which has been generated through the aforementioned process.

Genetically modified plants of the invention are capable of self-pollinating or crosspollinating with other plants of the same species so that the foreign gene, carried in the germ line, can be inserted into or bred into agriculturally useful plant varieties.

The term “plant cell” as used herein refers to protoplasts, gamete producing cells, and cells which regenerate into whole plants. Accordingly, a seed comprising multiple plant cells capable of regenerating into a whole plant, is included in the definition of “plant cell.

As used herein, the term “plant” refers to either a whole plant, a plant part, a plant cell, or a group of plant cells, such as plant tissue, for example. Plantlets are also included within the meaning of “plant” Plants included in the invention are any plants amenable to transformation techniques, including angiosperms, gymnosperms, monocotyledons and dicotyledons.

The following Examples are provided to illustrate the present invention, and should not be construed as limiting thereof.

### EXAMPLE 1

The transformation and transgenic plant regeneration can be carried by any of the previously published methods which are within the knowledge of those skilled in the art (e.g., U.S. Pat. No. 6,486,384, U.S. Pat. No. 6,534,313, WO0055302, US20020073446). The content of these issued patents and published patent applications are herein incorporated in full by a reference. The transformation method is based on the introduction of nucleic acids into meristematic tissues derived from any suitable source including, but not limited to, shoot meristems and leaf base tissue.

Following introduction of the nucleic acids into the target meristematic cells, incubation without selection permits the meristematic tissue to proliferate, and allows the transformed tissue to become established. Subsequent application of a selection agent permits the transformed tissue to be selected. After the tissue is selected (generally through multiple transfers to fresh selection medium to insure that the tissue comprising the meristematic domes is uniformly transformed), plants are induced to develop by removing or reducing the levels of hormones (particularly auxins) in the culturing medium (i.e., transfer to regeneration medium, RM). Plants can also be induced earlier in the selection process and resulting plants can be screened for presence of a transgene in the germline, thereby reducing the amount of time the tissue is cultured.

### Plant Culture Media and Methods

Unless otherwise noted, terms are to be understood according to conventional usage by those of ordinary skill in the relevant art. In addition to the definitions of terms provided below, definitions of common terms in molecular biology may also be found in standard molecular biology books

### A. In Vitro Culture of Meristematic Transformed Plant Cells

Meristematic tissue is comprised of undifferentiated plant cells that are capable of repeated division to yield other meristematic cells as well as differentiated cells that elongate and further specialize to form structural tissues and organs of the plant. Mersitematic tissue for use in the transformation methods described herein may be obtained from the shoot apices of seedlings or plants, as well as leaf bases.

The media used for in vitro culture of shoot meristematic tissue to produce adventitious meristems and to regenerate transformed meristematic tissue contribute significantly to the successful production of fertile transgenic plants. In addition, selection of fast-growing tissue improves the long-term regenerability of the cultures.

### B. Meristem Proliferation Medium (MPM)

Meristematic tissue isolated from a plant (e.g., shoot apices) is cultured on MPM medium, which promotes a fast growth rate and proliferation of meristematic cells without promoting shoot and root formation. In addition, following DNA introduction into meristematic tissue, the transformed tissues are incubated on MPM for a time sufficient for individual transformed cells to proliferate, thereby ensuring that a sufficient number of progeny cells are produced from each transformation event to increase the likelihood that the initial transformation event leads to the regeneration of a plant containing transformed tissue.

MPM preferably has a low auxin/high cytokinin ratio. Auxin levels in MPM are typically about 0 mg/L (no auxin) to about 3.0 mg/L. The preferred levels are about 0 mg/L to about 0.5 mg/L. Cytokinin levels in MPM are typically about 1 mg/L to about 10 mg/L. For certain plant species, about 2 mg/L to about 4 mg/L are preferred. Cytokinins may improve regenerability and reduce the incidence of albinism. The optimal level of cytokinin (and particularly the optimal ratio of auxin to cytokinin) depends on the genotype and the species being transformed. Any well-known auxin or cytokinin may be used in MPM or regeneration medium (RM). Auxins include, but are not limited to, dichlorophenoxyacetic acid [2,4-D], dicamba, indoleacetic acid, picloran and naphthalenacetic acid. 2,4-D is preferred for plant species of certain monocots such as barley, wheat, turf grass, forage grass and oat. Cytokinins include, but are not limited to, 6-benzylaminopurine [BAP], kinetin, zeatin, zeatin riboside, and N⁶-(2-isopentenyl)adenine (2iP). BAP and 2iP are typically employed for certain monocots such as turfgrass, forage grass and barley plant regeneration and transformation, particularly BAP. A particular genotype or species may respond optimally to a specific phytohormone.

MPM-MC refers to the particular formulation of MPM used in certain aspects of the invention. MPM-MC is formulated with hormones as described above, and is supplemented with maltose and copper. MCM-MC contains copper generally at a concentration of at least 0.1 μM (the level in typical plant growth media, such as MS medium), and more typically at least 10-100 fold higher, i.e. from about 1 to about 10 μM. In certain formulations, MPM contains even higher levels of copper, for example up to about 50 μM. Optimal copper levels vary with the genotype and species. The term “copper” is intended to include any well-known nutritional source of copper for plant culture media, e.g., cupric sulfate.

In addition, MPM also includes a sugar/carbon source, generally at about 20 g/L to about 60 g/L, with about 30 g/L being typical. In MPM-MC maltose is the preferred carbon/sugar source, particularly for recalcitrant monocots although sucrose or other conventional carbon sources for plant tissue culture can also be used.

Maltose and elevated copper levels may be tested separately and in combination in various formulations of MPM to observe their effects on in vitro culture of adventitious meristems. According to previously issued patents and published patent applications and reports that in some barley and wheat genotypes, the combination of maltose and elevated copper levels was critical for the successful long-term proliferation of shoot meristematic tissue and dramatically improved the shoot meristem proliferation efficiency in some barley genotypes. The combination of maltose and copper was necessary to produce long-term regenerable meristematic tissues. In addition selection of fast-growing tissue was also important for obtaining long-term regenerative cultures.

MPM can optionally be supplemented with a conventional osmoticum for a short time (e.g., about 4 hours) prior to (and, optionally, for a short period after) microprojectile bombardment. For example, the MPM can be supplemented with equimolar mannitol and sorbitol to give a final concentration of 0.4 M. The prior art teach good results have also been obtained when such an osmoticum was not included in MPM prior to (or after) bombardment.

The methods and media described herein can be used to produce and maintain adventitious meristematic tissue for long periods of time. To maintain adventitious meristematic tissue, it is generally divided into smaller pieces (e.g., pieces of about 3 to 5 mm for barley or turfgrasses (e.g. red fescue)) and subcultured, i.e., transferred to fresh medium, at regular intervals to promote optimal growth rates.

If a selectable marker is used to select for transformed tissues, the meristematic tissues may be initially cultured after transformation without selection in order to allow for the proliferation of transformed cells in the absence of dead or dying cells resulting from the selection agent. The optimal period for proliferation without selection varies with the species. After this period, selection can be applied to select for transformed cells. Selection can be accomplished by adding a selection agent to the culture medium for which the foreign DNA in transformed cells confers resistance (assuming that a selectable marker is included on the foreign DNA). Putative transformants are identified by their faster growth on the selective medium relative to nontransformed tissue. Screenable and scorable markers (e.g., green fluorescent protein and β-glucuronidase) can also be used to identify transformed tissue.

Transformed tissues are generally maintained under light, the use of light reduces or eliminates the regeneration of albino plants and improves regenerability.

As used herein, “plant culture medium” refers to any medium used in the art for supporting viability and growth of a plant cell or tissue, or for growth of whole plant specimens. Such media commonly include defined components including, but not limited to: macronutrient compounds providing nutritional sources of nitrogen, phosphorus, potassium, sulfur, calcium, magnesium, and iron; micronutrients, such as boron, molybdenum, manganese, cobalt, zinc, copper, chlorine, and iodine; carbohydrates (preferably maltose for turfgrasses and forage grasses as well as barley and wheat, although sucrose may be better for some species); vitamins; phytohormones; selection agents (for transformed cells or tissues, e.g., antibiotics or herbicides); and gelling agents (e.g., agar, Bactoagar, agarose, Phytagel, Gelrite, etc.); and may include undefined components, including, but not limited to: coconut milk, casein hydrolysate, yeast extract, and activated charcoal. The medium may be either solid or liquid, although solid medium is preferred.

Any conventional plant culture medium can be used as a basis for the formulation of MPM and RM when appropriately supplemented as described herein. In addition to the plant culture media discussed in the Examples in U.S. Pat. No. 6,486,384 (e.g., MS medium and FHG medium), a number of such basal plant culture media are commercially available from Sigma (St. Louis, Mo.) and other vendors in a dry (powdered) form for reconstitution with water.

### C. Regeneration Medium.

“Regeneration medium” (RM) promotes differentiation of totipotent plant tissues into shoots, roots, and other organized structures and eventually into plantlets that can be transferred to soil. Auxin levels in regeneration medium are reduced relative to MPM or, preferably, auxins are eliminated. It is also preferable that copper levels are reduced (e.g., to levels common in basal plant culture media such as MS medium). It is preferable to include a cytokinin in RM, as cytokinins have been found to promote regenerability of the transformed tissue. However, regeneration can occur without a cytokinin in the medium. Typically, cytokinin levels in RM are from about 0 mg/L to about 4 mg/L. For barley and wheat, about 2 mg/L of a cytokinin is preferred, and the preferred cytokinin is BAP. RM also preferably includes a carbon source, preferably about 20 g/L to about 30 g/L, e.g., either sucrose or maltose (there is no preference for maltose for RM). Optionally, one may employ a conventional shooting medium to promote shoot regeneration from meristematic structures and/or a conventional rooting medium to promote root formation. For example, MS basal medium supplemented with IBA (e.g., 0.5 mg/L) can be used to induce root formation, if necessary. Root induction is preferred for corn but appears to generally be unnecessary with oat and barley. Depending upon the genotype, different levels of an auxin and cytokinin (i.e., a different auxin/cytokinin ratio) provide optimal results. Conventional shooting and rooting media are considered regeneration media. Any well-known regeneration medium may be used for the practice of the methods of the present invention (e.g., U.S. Pat. No. 5,998,207, U.S. Pat. No. 6,479,287,U.S. Pat. No. 6,242,257).

### D. Introduction of Nucleic Acids

As discussed above, a number of methods can be used to introduce nucleic acids into the meristematic cells, including particle bomardment. Particle bombardment has been employed for transformation of a number of plant species, including barley and corn, for example. Successful transformation by particle bombardment requires that the target cells are actively dividing, accessible to microprojectiles, culturable in vitro, and totipotent, i.e., capable of regeneration to produce mature fertile plants. As described herein, a meristematic tissue (including, but not limited to a vegetative shoot meristem, such as an apical meristem from primary or axillary shoots, or a young leaf base) is cultured in vitro to caused to formation of adventitious meristems, and the adventitious meristem cells are the target for bombardment.

Microprojectile bombardment can be accomplished at normal rupture pressures, e.g., at about 1100 psi, although lower rupture pressures can be used to reduce damage of the target tissue, e.g., about 600 to 900 psi. It has been found that meristematic tissues recover better from the tissue damage caused by bombardment than callus tissue, permitting higher rupture pressures to be used.

In addition to particle bombardment, conventional methods for plant cell transformation may be used, including but not limited to: (1) Agrobacterium-mediated transformation, (2) microinjection, (3) polyethylene glycol (PEG) procedures, (4) liposome-mediated DNA uptake, (5) electroporation, and (6) vortexing with or without silica fibers.

### EXAMPLES

Mature seeds of red fescue seeds cv. Dawson are soaked in 70% ethanol for 2-3 min, washed 3× with sterilized water, sterilized with 30% (v/v) bleach (5.25% sodium hypochlorite) for 20-30 min (depending on seed source), and rinsed again with sterilized water 3×. Sterilized seeds are germinated on MS basal medium with 2% sucrose for 3-4 days under light (50 μEinsteins) at 23° C. Shoot apices (10-15 mm), including the shoot apical meristem, leaf primordia, and leaf bases, are isolated from germinated seedlings and cultured on meristem proliferation media (MPM). The MPM is MS basal medium (M-5519, Sigma, St. Louis, Mo., USA) supplemented with 500 mg/L casein enzymatic hydrolysate (Sigma, St. Louis, Mo., USA), 3% sucrose, 2,4-dichlorophenoxyacetic acid (2,4-D) at levels of 0.3 or 0.5 mg/L, and 2.0 mg/L 6-benzylaminopurine (BAP).

### Transformation of Red Fescue Using Shoot Meristematic Cultures

The shoot apices are isolated from the germinated seedlings and cultured on the shoot meristem proliferation medium MPM, comprising: MS+2.0 mg/L BAP+0.5 mg/L 2,4-D+500 mg/L casein hydrolysate+30 g/L sucrose, pH 5.6-5.8, or on MPM-MC which is MPM in which 30 g/L maltose replaced the sucrose and the level of CuSO₄ in (0.1 μM) is increased fifty-fold to 5.0 μM.

Shoot meristematic cultures (SMCs) is cut and placed in the petri dishes containing MPM-MC. Two transformation experiments are conducted: 1) co-transformation with pAHC20 and pAHC15 (“Ubiquitin promoter-based vectors for high-level expression of selectable and/or screenable marker genes in monocotyledonous plants,” Transgenic Research, 5:1-6.) using the particle inflow gun; 2) pUbi1NPTII-1 and pAHC15, using the Bio-Rad PDS1000He device as described (Lemaux et al., (1996), “Bombardment-Mediated Transformation Methods for Barley,” Bio-Rad, US/EG Bulletin 2007: 1-6). Osmoticum treatment is applied in both experiments. Bombarded SMCs are grown on MPM-MC for the first 2-3 weeks. After that, transformed tissues are selected on MPM-MC containing 3-5 mg/L of bialaphos for bar and 40-50 mg/L of G418 for nptII. After 3-4 months on selection, putative transgenic tissues are obtained and transferred to MS plus 2.0 mg/L BAP to induce vegetative shoot development. Putative bar-transformed shoots are transferred to MS containing 3-5 mg/L bialaphos to induce root development. Putative nptII-transformed shoots are rooted on MS medium without adding G418. Putative transgenic plants are transferred to the greenhouse. Progeny from transgenic plants are also individually harvested.

DNA samples are isolated from leaf tissue of greenhouse-grown plants using a urea extraction method (Maize Genet Coop News Lett 63: 68), digested with Hind III and/or EcoR I for plants transformed with pAHC20, pAHC15, or pUBiINPTII-1, transferred to Zeta-Probe GT blotting membrane (Bio-Rad Laboratories, CA 94547) using downward alkaline blotting (Koetsier et al. (1993) Biotechniques 15: 260-262) and hybridized with 0.6 kb fragment of bar and 1.8 kb of uidA using manufacture's instructions (Instruction Manual, Zeta-Probe GT Blotting Membranes). After washing, the blot is exposed to Kodak BioMax MS film (Fisher Scientific, IL).

Mature T₁ and T₂ seeds are harvested, surface-sterilized, and germinated on MS basal medium. Portions of young roots from each germinated seedlings are tested for GUS expression by histochemical staining with x-gluc (Jefferson et al. (1987) EMBO J 6: 3901-3907). Germinated seedlings are then transferred to MS medium with either 3-5 mg/L bialaphos to test for PAT (phosphinothricin acetyltransferase, product of bar), or 40-50 mg/L G418 for NPTII. An additional test for expression of herbicide resistance in greenhouse-grown plants involved leaf painting with 1% Basta solution (Hoechest AG, Frankfurt, Germany); plants are scored seven days after herbicide application. Chi-square analysis is applied to the segregation ratios for transgene expression in progeny. For analysis of physical transmission of bar and uidA, genomic DNA samples isolated from progeny plants are examined by PCR. Further description of the process of plant regeneration, genetic transformation as well as transgenic plant regeneration and the molecular and biochemical characterization of a transgenic plant can be found in issued patents and published patent applications (e.g., U.S. Pat. No. 6,486,384).

The complete disclosures of all publications that are cited herein are hereby incorporated by reference as if individually incorporated. It is also understood that, given the limitations of the state of the art, occasional sequence errors or deletions may occur without affecting the usefulness of the data presented. Various modifications and alterations of this invention will become apparent to those skilled in the art without departing from the scope and spirit of this invention, and it should be understood that this invention is not to be unduly limited to the illustrative embodiments set forth herein, but rather is to be construed to be of spirit and scope defined by the appended claims.

Table (1). List of genes and fragments thereof that are part of the invention: a gene encoding glucose oxidase, a gene encoding citrate synthase, a gene encoding A-9 desaturase, a gene encoding A-11 desaturase, a gene encoding a plant homolog of the neutrophil NADPH oxidase, a gene encoding bacteriopsin, a gene encoding an antiviral protein, a gene encoding cytokinin oxidase, a cytokinin regulated gene, an auxin-regulated gene, a gene encoding oxalate oxidase, a gene encoding cholesterol oxidase, a gene encoding a dehydration protein, a gene encoding an antimicrobial CEMA and/or CEMA-related peptide, acquired resistant genes, a gene encoding Bacillus thuringiensis δ-endotoxin, a gene encoding synthetically-modified B. thuringiensis a gene encoding δ-endotoxins, a gene encoding Bacillus thuringiensis Cry1C, a gene encoding an insecticidal protein toxins from Photorhabdus, a gene encoding glyphosate resistant EPSP synthase, a gene encoding protoporphyrinogen oxidase, a gene encoding thiroedoxin, anti-apoptosis gene, a BOS1 gene, a gene encoding a protein toxic to diabrotica insects, a gene encoding Bacillus thuringiensis delta-endotoxin, a gene encoding Bacillus thuringiensis CryET33 and CryET34 proteins, a gene encoding resistant 5-enolpyruvyl-3-phosphoshikimate synthetase, a glyphosate resistant gene, glufosinate resistant gene, a gene associated with nucleotide triphosphate transport, a gene encoding molybdenum cofactor sulfurase, a gene encoding an enzyme of the glycine betaine biosynthetic pathway, a gene encoding a dehydration regulation gene, a DIMBOA biosynthetic gene, 13-glucosidase gene, tryptophan gene, a gene encoding P450 reductase or cytochrome P450 enzyme, a bacterial mannitol-1-P dehydrogenase, a gene encoding an enzyme catalyzing the production of a polyol, a gene encoding an mt1D gene, a gene encoding anthranilate synthase, Bonsai gene, a gene encoding phospholipid binding protein, a gene encoding Bax inhibitor-1, a gene encoding metabolite transporter, a gene encoding molybdenum cofactor sulfurase, a gene encoding polyhydroxyalkanoate (e.g., 3-hydroxyacyl-acyl carrier protein thioesterase gene), an alcohol dehydrogenase gene, glutathione reductase gene, dehydroascorbate reductase gene, monodehydroascorbate reductase gene, a gene encoding mitochondrial alternative oxidase, a gene encoding NADH oxidase, a gene encoding NADPH oxidase, a gene encoding heat shock protein, a gene which reduces bioactive GA levels and the height of a plant, dwarfism genes, an acetohydroxyacid synthase gene, NIM1 genes, an amine oxidase gene, a gene which encodes phytochrome A, a gene which allows introducing proximity-conditional dwarfing to plants allowing them to be grown at high densities while maintaining good yields, gene which expresses a reactive oxygen species producing enzyme, peroxidase gene, polyphenol oxidase gene, germin-like oxalate oxidase, a gene encoding galactose oxidase, a gene encoding superoxide dismutatse, a gene encoding catalase, a gene encoding glutathion peroxidase, a gene encoding ascorbate peroxidase, a gene encoding oxalate decarboxylase, tfdA gene, a gene encoding a cold tolerance polypeptide from the Wcor410 family, a gene encoding a polypeptide having S-adenosylmethionine:methionine S-methyltransferase, gene encoding the enzyme isopentenyl transferase, choline monooxygenase, a gene encoding trehalose-6-phosphate, a gene encoding hydrogen peroxide-forming enzymes, glycolate oxidase, polyamine oxidase, copper amine oxidase, flavin amine oxidase, berberine Bridge Enzyme, choline oxidase, acyl coA oxidase, amino cyclopropane carboxylate oxidase (ACC oxidase), pyridoxamine-phosphate oxidase, sarcosine oxidase, sulfite oxidase, and methyl sterol oxidase, Superoxide-forming: aldehyde oxidase, xanthine Oxidase, and NADPH Oxidase (respiratory burst enzyme homolog), large subunit (GP91) (These additional genes and functional fragments are listed in Table 1). all of the listed references for these genes and fragments thereof as well as their full disclosure including nucleic acid and amino acid sequences are incorporated by reference.

The full contents of these references including nucleic acid sequences and amino acid sequences are also part of this invention. Therefore, during the prosecution of this patent or any related patent application which claims priority date to this patent application, the inventor will present some of these sequences in any of these patents as part of invention. The complete content of these patents are incorporated herein by a reference.

U.S. Pat. No. 5,780,709, WO03100032, US20020152499, WO9904012, EP1209227,

### US20020152499,

U.S. Pat. No. 6,441,275 Production of pathogen resistant plants

U.S. Pat. No. 6,403,861 Production of pathogen resistant plants

U.S. Pat. No. 6,380,461 Production of pathogen resistant plants

U.S. Pat. No. 6,380,460 Production of pathogen resistant plants

U.S. Pat. No. 6,376,748 Production of pathogen resistant plants

U.S. Pat. No. 6,303,326 Insect salivary enzyme triggers systemic resistance

U.S. Pat. No. 6,166,291 Production of pathogen resistant plants

U.S. Pat. No. 6,054,318 Expression of the glucose oxidase gene in transgenic organisms

EP0746616A4 EXPRESSION OF THE GLUCOSE OXIDASE GENE IN TRANSGENIC ORGANISMS

EP0746616A1 EXPRESSION OF THE GLUCOSE OXIDASE GENE IN TRANSGENIC ORGANISMS

WO9521924A1 EXPRESSION OF THE GLUCOSE OXIDASE GENE IN TRANSGENIC ORGANISMS

U.S. Pat. No. 6,686,179 Fusion polypeptides of human serum albumin and a therapeutically active polypeptide

U.S. Pat. No. 6,677,505 Promoter inductible in plants, sequence incorporating same and resulting product

U.S. Pat. No. 6,624,139 Hypersensitive response elicitor-induced stress resistance

U.S. Pat. No. 6,555,732 Rac-like genes and methods of use

U.S. Pat. No. 6,277,814 Enhancement of growth in plants

U.S. Pat. No. 6,262,018 Hypersensitive response elicitor from Erwinia amylovora and its use

U.S. Pat. No. 6,228,644 Hypersensitive response elicitor from Erwinia amylovora, its use, and encoding gene

U.S. Pat. No. 6,175,059 Development of a novel gene delivery system through seed coating

U.S. Pat. No. 6,172,184 Hypersensitive response elicitor from Pseudomonas syringae and its use

U.S. Pat. No. 6,072,105 Insect-resistant transgenic eggplant and method of making

U.S. Pat. No. 5,516,671 Method of controlling plant pathogens

CA2183067AA EXPRESSION OF THE GLUCOSE OXIDASE GENE IN TRANSGENIC ORGANISMS

### EP0904396B1 PRODUCTION OF VANILLIN

EP1122316A1 PROCESS FOR OBTAINING TRANSGENIC PLANTS WHICH HAVE AN IMPROVED CAPACITY FOR THE UPTAKE OF NUTRIENTS AND TOLERANCE TO TOXIC COMPOUNDS WHICH ARE PRESENTS IN THE SOIL

WO9832843A2 COMPOSITIONS AND METHOD FOR MODULATION OF ALKALOID BIOSYNTHESIS AND FLOWER FORMATION IN PLANTS

WO0004133A1 AGROBACTERIUM-MEDIATED TRANSFORMATION OF TURFGRASS

US20010014467A1 Production of vanillin

U.S. Pat. No. 5,866,789 Fatty acid alteration by a 9 desaturase in transgenic plant tissue

### CA2084348AA FATTY ACID ALTERATION BY A 9 DESATURASE IN TRANSGENIC PLANT TISSUE

WO0004133A1 AGROBACTERIUM-MEDIATED TRANSFORMATION OF TURFGRASS

U.S. Pat. No. 5,656,466 Process for preparing virus-resistant transgenic plant
U.S. Pat. No. 5,633,155 Expression vector for phytolacca antiviral protein and process for preparing transgenic plant transformed therewith
EP0707069A3 Process for preparing virus-resistant transgenic plant
U.S. Pat. No. 6,015,942 Transgenic plants exhibiting heterologous virus resistance
U.S. Pat. No. 6,013,864 Plants resistant to infection by luteoviruses
U.S. Pat. No. 6,617,497 Cytokinin oxidase

U.S. Pat. No. 6,229,066 Cytokinin oxidase

US20040031073A1 Method for modifying plant morphology, biochemistry and physiology

US20030163847A1 Reversible male sterility in transgenic plants by expression of cytokinin oxidase

WO03050287A3 METHOD FOR MODIFYING PLANT MORPHOLOGY, BIOCHEMISTRY AND PHYSIOLOGY COMPRISING EXPRESSION OF PLANT CYTOKININ OXIDASE

WO03050287A2 METHOD FOR MODIFYING PLANT MORPHOLOGY, BIOCHEMISTRY AND PHYSIOLOGY

US20030074698A1 Method for modifying plant morphology, biochemistry and physiology

U.S. Pat. No. 6,444,470 Transformation-enhancing compositions and methods of use

### WO0190388A1 GENE CONTROLLING SHOOT BRANCHING IN PLANTS

U.S. Pat. No. 6,225,529 Seed-preferred promoters

U.S. Pat. No. 5,972,660 Human hydroxypyruvate reductase

U.S. Pat. No. 5,910,444 Plants in which the expression of S-adenosylhomocysteine hydrolase gene is inhibited

### WO03000898A1 PLANT GENES INVOLVED IN DEFENSE AGAINST PATHOGENS

U.S. Pat. No. 6,613,959 Transgenic plants expressing a MAPKKK protein kinase domain

U.S. Pat. No. 6,548,743 Transgenic plants expressing a dual-specificity MAPK phosphatase and uses thereof

U.S. Pat. No. 6,043,410 Strawberry fruit promoters for gene expression

U.S. Pat. No. 5,981,841 Early seed 5′ regulatory sequence

U.S. Pat. No. 6,229,065 Production of plants resistant to attacks by Sclerotinia sclerotiorum by the introduction of a gene encoding an oxalate oxidase

U.S. Pat. No. 6,072,050 Synthetic promoters

U.S. Pat. No. 5,981,842 Production of water stress or salt stress tolerant transgenic cereal plants

CA2354228AA PLANTS AND SEEDS CONTAINING SYNTHETIC PROMOTERS

U.S. Pat. No. 5,866,778 Newly characterized oxalate and uses therefore.

U.S. Pat. No. 6,500,617 Optimization of pest resistance genes using DNA shuffling

U.S. Pat. No. 5,763,245 Method of controlling insects

### WO03076597A2 NUCLEIC ACID SEQUENCES ENCODING THE BOS1 GENE AND PROMOTER FROM ARABIDOPISIS THALIANA AND USES THEREOF

U.S. Pat. No. 6,281,411 Transgenic monocots plants with increased glycine-betaine content

U.S. Pat. No. 6,310,271 Polynucleotides encoding choline monooxygenase and plants transformed therewith

U.S. Pat. No. 6,395,966 Fertile transgenic maize plants containing a gene encoding the pat protein

U.S. Pat. No. 6,329,574 High lysine fertile transgenic corn plants

U.S. Pat. No. 6,326,527 Method for altering the nutritional content of plant seed

U.S. Pat. No. 6,271,016 Anthranilate synthase gene and method of use thereof for conferring tryptophan overproduction

U.S. Pat. No. 6,281,412 Method for creating osmotic-pressure-tolerant plant

U.S. Pat. No. 6,118,047 Anthranilate synthase gene and method of use thereof for conferring tryptophan overproduction

WO9826081A1 TRANSGENIC PLANTS HAVING INCREASED FREEZING AND CHOLINE TOLERANCE

U.S. Pat. No. 5,633,153 Aldehyde dehydrogenase selectable markers for plant transformation

U.S. Pat. No. 6,703,539 Method and compositions for modifying levels of secondary metabolic compounds in plants

U.S. Pat. No. 6,483,013 Method for agrobacterium mediated transformation of cotton

U.S. Pat. No. 6,423,838 Gene for proline transporter in rice

U.S. Pat. No. 6,399,861 Methods and compositions for the production of stably transformed, fertile monocot plants and cells thereof

EP1015599A2 POLYNUCLEOTIDES ENCODING CHOLINE MONOOXYGENASE AND PLANTS TRANSFORMED THEREWITH

U.S. Pat. No. 6,025,545 Methods and compositions for the production of stably transformed, fertile monocot plants and cells thereof

U.S. Pat. No. 5,990,390 Methods and compositions for the production of stably transformed, fertile monocot plants and cells thereof

U.S. Pat. No. 5,874,265 Methods and compositions for the production of stably transformed fertile monocot plants and cells thereof

WO9830702A3 POLYNUCLEOTIDES ENCODING CHOLINE MONOOXYGENASE AND PLANTS TRANSFORMED THEREWITH

WO9830702A2 POLYNUCLEOTIDES ENCODING CHOLINE MONOOXYGENASE AND PLANTS TRANSFORMED THEREWITH

CA2276612AA POLYNUCLEOTIDES ENCODING CHOLINE MONOOXYGENASE AND PLANTS TRANSFORMED THEREWITH

U.S. Pat. No. 5,780,709 Transgenic maize with increased mannitol content

U.S. Pat. No. 6,331,660 Maize DIMBOA biosynthesis genes

WO9840505 μl MAIZE DIMBOA BIOSYNTHESIS GENES

US20030188344A1 Compositions and methods for agrobacterium transformation of plants

### WO9506128A2 FERTILE, TRANSGENIC MAIZE PLANTS AND METHODS FOR THEIR PRODUCTION

US20010052140A1 Methods of selection and development of plants having improved root quality and root lodging resistance

US20030213010A1 Transgenic high tryptophan plants

US20030097677A1 Transgenic high tryptophan plants

U.S. Pat. No. 6,433,249 Use of β-glucosidase to enhance disease resistance and resistance to insects in crop plants

### WO0173091A2 METHODS OF SELECTION AND DEVELOPMENT OF PLANTS HAVING IMPROVED ROOT QUALITY AND ROOT LODGING RESISTANCE

### WO0028012A2 THE USE OF 13-GLUCOSIDASE TO ENHANCE DISEASE RESISTANCE AND RESISTANCE TO INSECTS IN CROP PLANTS

WO0000585A2 MAIZE CYTOCHROME P450 MONOOXYGENASE cDNA (CYP71C3v2)

WO0000502A1 MAIZE CYTOCHROME P450 MONOOXYGENASE cDNA (CYP71C3v2)

U.S. Pat. No. 5,569,597 Methods of inserting viral DNA into plant material
U.S. Pat. No. 6,020,288 Methods and compositions for enhancing cytochrome P450 in plants
U.S. Pat. No. 6,613,961 Plants capable of metabolizing drugs and use thereof

U.S. Pat. No. 5,948,955 Transgenic plants having altered anthocyann levels

US20030166255A1 Cytochrome P450s and uses thereof

U.S. Pat. No. 6,610,908 Manipulation of lignin composition in plants using a tissue-specific promoter

U.S. Pat. No. 6,605,469 Nucleic acid molecule encoding a cytochrome P450 hydroxylase in brassinosteroid biosynthesis in plants

WO02072758A3 CYTOCHROME P450S AND USES THEREOF

WO02072758A2 CYTOCHROME P450S AND USES THEREOF

CA2440278AA CYTOCHROME P450S AND USES THEREOF

U.S. Pat. No. 6,410,718 Materials and methods for the modification of plant lignin content

U.S. Pat. No. 6,204,434 Materials and methods for the modification of plant lignin content

U.S. Pat. No. 6,020,288 Methods and compositions for enhancing cytochrome P450 in plants

U.S. Pat. No. 5,952,486 Materials and methods for the modification of plant lignin content

U.S. Pat. No. 5,753,507 Plant geraniol/nerol 10-hydroxylase and DNA coding therefor

U.S. Pat. No. 6,723,897 Methods for controlling gibberellin levels

U.S. Pat. No. 6,673,537 Promoters from chlorella virus genes providing for expression of genes in prokaryotic and eukaryotic hosts

U.S. Pat. No. 6,670,396 Aspirin-triggered lipid mediators

U.S. Pat. No. 6,649,814 Cytochrome P450 monooxygenases

U.S. Pat. No. 6,642,439 Basidiomycete manganese peroxidase gene-transferred plant and a method for removing an environmental contaminant using the same

U.S. Pat. No. 6,639,130 Plant sterol reductases and uses thereof

US20030166202A1 P450 Monooxygenases of the cyp79 family

US20030150025A1 DAS5, a P450 protein involved in the brassinosteroid biosynthesis pathway in plants

US20030041346A1 Cytochrome P450 monooxygenases

U.S. Pat. No. 6,517,856 Insecticide resistance assay

U.S. Pat. No. 6,500,639 DNA shuffling to produce nucleic acids for mycotoxin detoxification

U.S. Pat. No. 6,489,538 Method for regulation of plant lignin composition

U.S. Pat. No. 6,444,874 Hydroperoxide lyase gene from maize and methods of use

U.S. Pat. No. 6,441,272 Modification of lignin content and composition in plants

EP1209227A3 Cytochrome P450 hydroxylase, an important enzyme in brassinosteroid biosynthesis of plants

U.S. Pat. No. 6,392,123 Female-preferential promoters isolated from maize and wheat

U.S. Pat. No. 6,300,544 Cytochrome P450 monooxygenases

JP2001231560A2 CYTOCHROME P450 GENE HIGHLY EXPRESSED IN INCOMPATIBLE INTERACTION

WO0151622A3 P450 MONOOXYGENASES OF THE CYP79 FAMILY

U.S. Pat. No. 6,252,140 Promoters from chlorella virus genes providing for expression of genes in prokaryotic and eukaryotic hosts

JP2001069989A2 INDUCTIVE PROMOTER REGION ORIGINATING FROM TOBACCO

U.S. Pat. No. 6,133,417 Cytochrome P-450 monooxygenases

U.S. Pat. No. 6,121,512 Cytochrome P-450 constructs and method of producing herbicide-resistant transgenic plants

WO0053771A1 GENETIC MANIPULATION OF ISOFLAVONOIDS

CA2362440AA GENETIC MANIPULATION OF ISOFLAVONOIDS

WO0009720A3 CYTOCHROME B5 FROM PETUNIA

WO0009720A2 CYTOCHROME B5 FROM PETUNIA

EP0970191A2 CYTOCHROME P450 MONOOXYGENASES

U.S. Pat. No. 5,981,837 Method for regulation of plant lignin composition

U.S. Pat. No. 5,965,387 Promoter

U.S. Pat. No. 5,952,545 Nucleic acid molecules encoding cytochrome P450-type proteins involved in the brassinosteroid synthesis in plants

U.S. Pat. No. 5,882,851 Cytochrome P-450 monooxygenases

U.S. Pat. No. 5,861,487 Genetic sequences encoding flavonoid pathway enzymes and uses therefor

EP0889963A1 NUCLEIC ACID MOLECULES ENCODING CYTOCHROME P450-TYPE PROTEINS INVOLVED IN THE BRASSINOSTEROID SYNTHESIS IN PLANTS

WO9840470A3 CYTOCHROME P450 MONOOXYGENASES

U.S. Pat. No. 5,824,859 Blue light photoreceptors and methods of using the same

WO9840470A2 CYTOCHROME P450 MONOOXYGENASES

U.S. Pat. No. 5,759,517 Hemoglobins as drug delivery agents

WO9735986A1 NUCLEIC ACID MOLECULES ENCODING CYTOCHROME P450-TYPE PROTEINS INVOLVED IN THE BRASSINOSTEROID SYNTHESIS IN PLANTS

CA2250119AA NUCLEIC ACID MOLECULES ENCODING CYTOCHROME P450-TYPE PROTEINS INVOLVED IN THE BRASSINOSTEROID SYNTHESIS IN PLANTS

U.S. Pat. No. 5,569,832 Genetic sequences encoding flavonoid pathway enzymes and uses therefor

U.S. Pat. No. 5,563,324 Transgenic plants with altered polyol content

### WO9219731A1 TRANSGENIC PLANTS WITH ALTERED POLYOL CONTENT

U.S. Pat. No. 5,554,798 Fertile glyphosate-resistant transgenic corn plants
U.S. Pat. No. 6,153,811 Method for reduction of transgene copy number
US20020194639A1 Bonsai, a phospholipid binding protein, is required for thermal tolerance in arabidopsis

WO0200697A3 BONSAI, A PHOSPHOLIPID BINDING PROTEIN, IS REQUIRED FOR THERMAL TOLERANCE IN ARABIDOPSIS

U.S. Pat. No. 6,586,658 Modification of fatty acid metabolism in plants

U.S. Pat. No. 5,650,555 Transgenic plants producing polyhydroxyalkanoates

U.S. Pat. No. 6,103,956 Polyhydroxyalkanoate synthesis in plants

U.S. Pat. No. 6,091,002 Polyhydroxyalkanoates of narrow molecular weight distribution prepared in transgenic plants

U.S. Pat. No. 5,958,745 Methods of optimizing substrate pools and biosynthesis of poly-β-hydroxybutyrate-co-poly-β-hydroxyvalerate in bacteria and plants

U.S. Pat. No. 5,942,660 Methods of optimizing substrate pools and biosynthesis of poly-β-hydroxybutyrate-co-poly-β-hydroxyvalerate in bacteria and plants

U.S. Pat. No. 6,709,848 Methods for isolating polyhydroxyalkanoates from plants

U.S. Pat. No. 6,689,589 Biological systems for manufacture of polyhydroxyalkanoate polymers containing 4-hydroxyacids

U.S. Pat. No. 6,316,262 Biological systems for manufacture of polyhydroxyalkanoate polymers containing 4-hydroxyacids

U.S. Pat. No. 6,143,561 DNA encoding plastid pyruvate dehydrogenase and branched chain oxoacid dehydrogenase components

U.S. Pat. No. 6,083,729 Methods for isolating polyhydroxyalkanoates from plants

U.S. Pat. No. 6,593,116 Transgenic microbial polyhydroxyalkanoate producers

U.S. Pat. No. 6,576,450 Polyhydroxyalkanoate production from polyols

U.S. Pat. No. 6,528,706 Polyhydroxybutyrate polymerase

U.S. Pat. No. 6,329,183 Polyhydroxyalkanoate production from polyols

U.S. Pat. No. 6,228,623 Polyhydroxyalkanoates of narrow molecular weight distribution prepared in transgenic plants

U.S. Pat. No. 6,022,729 Granule-associated proteins and methods for their use in polyhydroxyalkanoate biosynthesis

U.S. Pat. No. 5,959,179 Method for transforming soybeans

WO04006657A1 TRANSGENIC PLANTS USED AS A BIOREACTOR SYSTEM

US20030233677A1 Modification of fatty acid metabolism in plants

U.S. Pat. No. 6,600,029 Metabolic engineering of polyhydroxyalkanoate monomer synthases

US20030004299A1 Production of polyhydroxyalkanoates

U.S. Pat. No. 6,495,348 Mitomycin biosynthetic gene cluster

US20020187530A1 Biological systems for manufacture of polyhydroxyalkanoate polymers containing 4-hydroxyacids

U.S. Pat. No. 6,475,734 Polyhydroxyalkanoate synthase genes

U.S. Pat. No. 6,448,473 Multigene expression vectors for the biosynthesis of products via multienzyme biological pathways

U.S. Pat. No. 6,368,836 Method of decolorizing or deodorizing polyhydroxyalkanoates from biomass with ozone

US20020040485A1 TRANSGENIC SYSTEMS FOR THE MANUFACTURE OF POLY(2-HYDROXY-BUTYRATE-CO-3-HYDROXYHEXANOATE)

U.S. Pat. No. 6,207,217 Animal nutrition compositions

U.S. Pat. No. 6,143,952 Modified pseudomonas oleovorans phaC1 nucleic acids encoding bispecific polyhydroxyalkanoate polymerase

U.S. Pat. No. 6,127,606 Method of using transactivation proteins to control expression in transgenic plants

U.S. Pat. No. 5,899,339 Process for recovering polyhydroxyalkanoates using centrifugal fractionation

U.S. Pat. No. 5,849,854 Process for recovering polyhydroxyalkanotes using air classification

U.S. Pat. No. 5,801,027 Method of using transactivation proteins to control gene expression in transgenic plants

U.S. Pat. No. 5,610,041 Processes for producing polyhydroxybutyrate and related polyhydroxyalkanoates in the plastids of higher plants

### WO03100032A2 DWARFISM GENES AND DWARF PLANTS

US20030226177A1 Dwarfism genes and dwarf plants
U.S. Pat. No. 5,945,579 Modification of crop plant architecture to enhance yield by causing proximity-conditional dwarfing to control shade avoidance reactions

WO02052923A2 LONG DAY PLANTS CHARACTERIZED BY ALTERED FLOWERING RESPONSE TO DAY LENGTH UNDER COMMERCIAL AND GARDEN CONDITIONS AND METHODS OF GENERATING SAME

U.S. Pat. No. 6,303,846 Gene encoding oxalate decarboxylase from aspergillus phoenices
U.S. Pat. No. 6,297,425 Gene encoding oxalate decarboxylase from aspergillus phoenices
US20040068769A1 Enhancement of freezing tolerance in transgenic plants
U.S. Pat. No. 6,452,068 Chemical inducible promoters used to obtain transgenic plants with a silent marker

U.S. Pat. No. 5,689,042 Transgenic plants with altered senescence characteristics

U.S. Pat. No. 6,359,197 Transgenic plants with altered senescence characteristics

U.S. Pat. No. 6,723,896 Inducible site-specific recombination for the activation and removal of transgenes in transgenic plants

U.S. Pat. No. 6,268,552 Transgenic seedless fruit comprising AGL or GH3 promoter operably linked to isopentenyl transferase or tryptophan monooxygenase coding DNA

U.S. Pat. No. 6,063,985 Chemical inducible promoter used to obtain transgenic plants with a silent marker

EP1384785A1 Method for producing transgenic plant using an auxin synthesis gene as a positive selectable marker gene

U.S. Pat. No. 6,667,428 High efficiency plant transformation of brassica oleracea

U.S. Pat. No. 6,407,312 Esr1-A plant gene that can promote plant regeneration and transformation

EP0999738A4 TRANSGENIC SEEDLESS FRUIT AND METHODS

U.S. Pat. No. 6,329,570 Cotton modification using ovary-tissue transcriptional factors

U.S. Pat. No. 6,268,547 Transformation of wheat with the cyanamide hydratase gene

EP0999738A1 TRANSGENIC SEEDLESS FRUIT AND METHODS

U.S. Pat. No. 5,767,374 Plants with modified flowers seeds or embryos

U.S. Pat. No. 5,633,441 Plants with genetic female sterility

U.S. Pat. No. 5,177,307 Compositions and methods for modulation of endogenous cytokinin levels

U.S. Pat. No. 6,686,516 Expression of trehalose 6-phosphate synthase in plant plastids

U.S. Pat. No. 6,664,387 Expression cassette and plasmid for strong constitutive gene expression and the use thereof

EP1253202A1 Expression cassette comprising the Arabidopsis thaliana trehalose-6-phosphate synthase 3 (TPS3) promoter for strong constitutive gene expression

U.S. Pat. No. 6,130,368 Transgenic plants producing trehalose

EP0763118A1 TRANSGENIC PLANTS PRODUCING TREHALOSE

1. A method for genetically modifying a turfgrass plant cell such that a plant, produced from said cell, is characterized as having modulated brassinolide activity as compared with a wild-type plant, said method comprising:
introducing at least one exogenous BAS1 polynucleotide, into a plant cell to obtain a transformed plant cell; and
growing the transformed plant cell under conditions which permit expression of BAS1 gene product,
thereby producing a plant having modulated brassinolide activity, wherein the plant cell is a turfgrass cell wherein said genetically modified plant further comprising a drought resistance, salt resistance, and insect resistance; wherein the BAS 1 is a polypeptide having the amino acid sequence of SEQ ID NO: 2.

2. The method of claim 1, wherein the transformed plant is dwarf and dark-green.

3. The method of claim 2, wherein the plant is a bentgrass, kentucky bluegrass, bermudagrass, zosiagrass, fescues, and ryegrass.

4. The method of claim 1, wherein said modulation is suppressed brassinolide activity.

5. The method of claim 1, wherein said suppression of brassinolide activity is achieved by overexpression of BAS1 in the plant; wherein the BAS 1 is a polypeptide having the amino acid sequence of SEQ ID NO: 2.

6. The method of claim 1, further comprising introducing at least one exogenous polynucleotide comprising a chibi2 structural gene into the plant cell to obtain the transformed plant cell and growing the transformed plant cell under conditions which permit expression of the chibi2 gene product, wherein the plant cell is a turfgrass plant origin.

7. A method of producing a genetically modified turfgrass plant characterized as having dwarf adult stature with dark green foliage, said method comprising:
contacting a plant cell with a vector containing an exogenous nucleic acid sequence comprising at least one structural gene encoding a BAS 1 polypeptide, said gene being operably associated with a regulatory sequence that causes overexpression of the gene, to obtain a transformed plant cell;
producing a plant from said transformed plant cell; and
selecting a plant exhibiting said dwarf adult stature with dark green foliage; wherein the BAS 1 is a polypeptide having the amino acid sequence of SEQ ID NO: 2.

8. The method of claim 7, wherein the contacting is by physical means.

9. The method of claim 7, wherein the contacting is by chemical means.

10. The method of claim 7, wherein the plant cell is selected from the group consisting of protoplasts, gamete producing cells, and cells that regenerate into a whole plant.

11. The method of claim 7, wherein the regulatory sequence comprises a constitutive promoter.

12. The method of claim 7, wherein the regulatory sequence comprises an inducible promoter.

13. A plant produced by the method of claim 7.

14. Plant tissue derived from a plant produced by the method of claim 7; wherein the plant issue a nucleic acid that was introduced to a parent.

15. A seed derived from a plant produced by the method of claim 7; wherein the seed contains a nucleic acid that was introduced to a parent.

16. An embryo cell from the transgenic plant produced by the method of claim 7; wherein the embry cell contains a nucleic acid that was introduced to a parent.

17. A callus cell from the transgenic plant produced by the method of claim 7; wherein the callus cell contains a nucleic acid that was introduced to a parent.

18. A protoplast from the transgenic plant produced by the method of claim 7; wherein the protoplast contains a nucleic acid that was introduced to a parent.

19. A method of producing genetically transformed turfgrass plant that is resistant to a disease,
comprising introducing into the genome of a turfgrass plant cell to obtain a transformed plant cell, a nucleic acid sequence comprising an expression control sequence operably linked to a polynucleotide encoding BAS 1 polypeptide.
wherein said transgenic plant further comprising a drought resistance, salt resistance, and insect resistance; wherein the BAS 1 is a polypeptide having the amino acid sequence of SEQ ID NO: 2.

20. The method of claim 19, wherein said expression control sequence targets expression to a plant part selected from the group consisting of leaves, roots, shoots, and stems.


SEQUENCE LISTING (2 sequences)

SEQ ID NO: 1 - DNA, 1563 bp, ARABIDOPISIS
  gene (1)..(1563)
atggaggaagaaagtagcagctggttcattccaaaggttcttgttctgtctgtaatcttaagtcttgtaatagtgaagggtatgtctctgttatggtggagaccaagaaagattgaagaacatttctctaaacaaggaattcgaggtcctccttatcatttcttcatcggaaatgttaaagaacttgttggaatgatgcttaaagcttcttctcatcctatgcctttctctcacaatattcttcctagagttctctctttttaccatcactggagaaaaatctacggtgctacatttctggtttggttcggtccaactttccggttaacggtagccgatcctgatttgatcagagagatcttctctaagtctgagttctacgagaagaatgaagctcaccctttggttaaacaacttgaaggcgatggactacttagtctcaaaggtgaaaaatgggctcatcatcgaaaaatcattagccctacttttcatatggagaatcttaagttgcttgtaccagttgtgttgaagagtgtgactgatatggtggataaatggtccgataagttatcagaaaacggtgaagttgaggtagatgtctatgagtggtttcagattttgactgaagatgttattagtagaacagcttttggaagtagctatgaagatggtcgagcagtttttcgacttcaagctcaacaaatgcttctttgtgctgaagcttttcaaaaagtcttcattcctggctatagattttttccgacaagagggaatttgaagtctcggaagttagacaaggagataaggaagtcgttgttgaagctgatagagcggcggagacaaaacgctatagatggagaaggggaagaatgtaaggagccggcggcgaaggatttgttgggattaatgattcaggcaaagaatgtgacggttcaggacattgtggaggagtgtaaaagctttttcttcgccgggaaacagacaacttctaatctgctgacgtggacgaccatcttgctatccatgcacccggagtggcaggccaaagcacgtgatgaggtcctcagggtctgcggctcacgtgatgtccctaccaaggaccatgtcgttaagcttaaaacgttgagtatgatcttgaacgagtctttaaggttgtatccaccaatagtagctacgattcgacgcgctaaatcggatgtgaagctaggagggtacaaaatcccatgtggcacggagcttctaatcccaatcatagcggtccatcatgaccaagccatttggggtaatgacgtgaacgaattcaatccagctcggtttgcggatggagtgccgcgtgctgccaaacaccccgttggcttcataccgtttggcctcggagttcgtacatgcattggtcagaatcttgctatacttcaggccaaattgacactcgctgtaatgatccaacgcttcacctttcacttggctcctacttatcagcatgcacctaccgtccttatgttgctttatcctcaacatggtgcaccaatcaccttccggagattgaccaatcatgaggattga

SEQ ID NO: 2 - protein, 520 aa, ARABIDOPISIS
  PEPTIDE (1)..(520)
MEEESSSWFIPKVLVLSVILSLVIVKGMSLLWWRPRKIEEHFSKQGIRGPPYHFFIGNVKELVGMMLKASSHPMPFSHNILPRVLSFYHHWRKIYGATFLVWFGPTFRLTVADPDLIREIFSKSEFYEKNEAHPLVKQLEGDGLLSLKGEKWAHHRKIISPTFHMENLKLLVPVVLKSVTDMVDKWSDKLSENGEVEVDVYEWFQILTEDVISRTAFGSSYEDGRAVFRLQAQQMLLCAEAFQKVFIPGYRFFPTRGNLKSRKLDKEIRKSLLKLIERRRQNAIDGEGEECKEPAAKDLLGLMIQAKNVTVQDIVEECKSFFFAGKQTTSNLLTWTTILLSMHPEWQAKARDEVLRVCGSRDVPTKDHVVKLKTLSMILNESLRLYPPIVATIRRAKSDVKLGGYKIPCGTELLIPIIAVHHDQAIWGNDVNEFNPARFADGVPRAAKHPVGFIPFGLGVRTCIGQNLAILQAKLTLAVMIQRFTFHLAPTYQHAPTVLMLLYPQHGAPITFRRLTNHED